(19) United States
(12) Patent Application Publication
(10) Pub. No.: US 2024/0261297 A1
(43) Pub. Date: Aug. 8, 2024

(54) ANTI-CDK INHIBITORS FOR CANCER TREATMENT

(71) Applicant: St. John's Cancer Institute, Santa Monica, CA (US)

(72) Inventor: Venkata M. Yenugonda, Woodland Hills, CA (US)

(73) Assignee: St. John's Cancer Institute, Santa Monica, CA (US)

(21) Appl. No.: 18/561,888

(22) PCT Filed: May 17, 2022
(86) PCT No.: PCT/US2022/029564

§ 371 Date: Nov. 17, 2023

Related U.S. Application Data
(60) Provisional application No. 63/190,974, filed on May 20, 2021.
(60) Provisional application No. 63/210,795, filed on Jun. 15, 2021.

(51) Int. Cl. A61K 31/5377; A61K 31/506; A61P 35/00; C07D 471/04

(52) U.S. Cl. CPC A61K 31/5377; A61K 31/506; A61P 35/00; C07D 471/04

(57) ABSTRACT

Disclosed herein are compounds and methods for treating cancer. The compound may have a formula according to Formula I, or a pharmaceutically acceptable salt thereof.

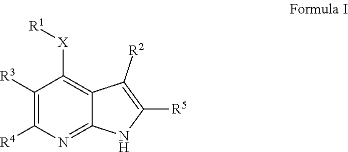

In some examples, the compounds are inhibitors of one or more cyclin-dependent kinases.

### CROSS REFERENCE TO RELATED APPLICATION

This application claims the benefit of the earlier filing dates of U.S. provisional patent application No. 63/190,974, filed May 20, 2021, and U.S. provisional patent application No. 63/210,795, filed Jun. 15, 2021, both of which are incorporated herein by reference in their entirety.

### FIELD

The present disclosure is directed to embodiments of cyclic dependent kinase inhibitor compounds and method embodiments for using the same to treat cancer.

### BACKGROUND

Deregulation of CDKs has been reported to cause unscheduled proliferation as well as genomic and chromosomal instability resulting in human cancer, and in addition to contribute to both cancer progression and aggressiveness. Additionally, many cancers are uniquely dependent on CDKs and hence are selectively sensitive to their inhibition. Cyclin dependent kinases (CDKs) are serine/threonine kinases that are activated when they bind to their respective cyclin units, and currently 20 CDKs and 25 cyclins have been identified. Members of the CDK family play an important role in regulating the cell cycle, and dysregulation of CDK function is a common finding in cancer, making them a potential anti-cancer target. Abnormal regulation of the Cyclin D-CDK4-/6-INK4-retinoblastoma protein (Rb) signaling pathway is among the most common aberrations found in many human cancers.

### SUMMARY

Disclosed herein is a compound according to Formula I

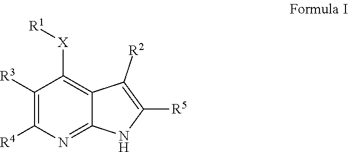

or a pharmaceutically acceptable salt thereof, wherein, R¹ is aromatic, heterocycloaliphatic, or cycloalkyl, optionally substituted with one or more substituents; X is O, S, SO₂, CH₂, NH, NMe or a bond; R² is heteroaryl substituted with NR′₂ or OH and optionally further substituted with one or more additional substituents; each R′ independently is H, C₁₋₆alkyl or —C(O)CH₂N(C₁₋₆alkyl)₂; and each of R³, R⁴ and R⁵ independently is H or aliphatic.

The compound may have a formula according to one of formulas II, II-A, III, III-A, or IV, or a pharmaceutically acceptable salt thereof:

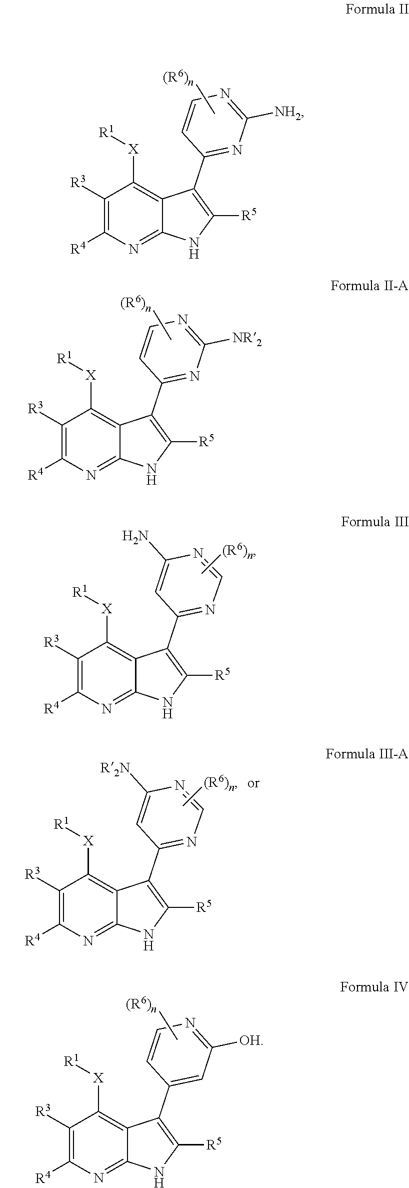

With respect to these formulas, n is 0, 1, or 2, each R⁶ independently is amino, OH, halogen, alkyl, haloalkyl, CO₂H, CO₂R, NO₂, CN, amide, sulfonic acid, sulfonamide, hydroxyalkyl, alkoxy, or heteroaliphatic, where R is aliphatic, and each R′ independently is H, C₁₋₆alkyl or —C(O)CH₂N(C₁₋₆alkyl)₂. In certain embodiments, the compound has a structure according to Formulas II or III.

In alternative embodiments, the compound has a formula according to formulas V or VI or a pharmaceutically acceptable salt thereof:

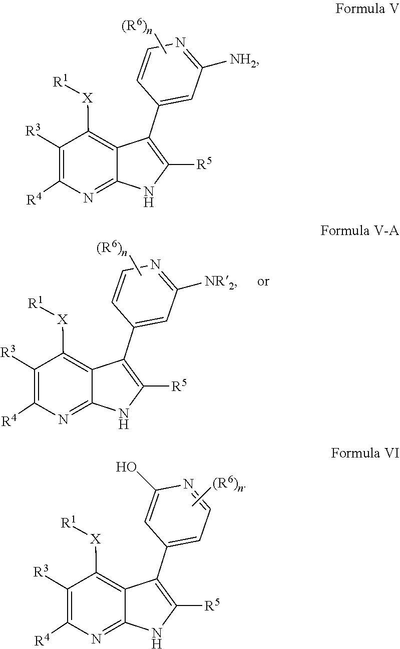

With respect to formulas V and VI, n is 0, 1, or 2, each R⁶ independently is amino, OH, halogen, alkyl, haloalkyl, CO₂H, CO₂R, NO₂, CN, amide, sulfonic acid, sulfonamide, hydroxyalkyl, alkoxy, or heteroaliphatic, where R is aliphatic, and each R′ independently is H, C₁₋₆alkyl or —C(O)CH₂N(C₁₋₆alkyl)₂. In certain embodiments, the compound has a structure according to Formulas V or VI.

In some embodiments, X is O. And/or in some embodiments, R¹ is phenyl, pyridinyl, pyrimidinyl, quinolinyl, imidazolyl, pyrazinyl, or furanyl. However, in alternative embodiments, R¹ is cycloalkyl, and may be a bridged cycloalkyl. In other embodiments, R¹ is heterocycloaliphatic.

In certain embodiments, the compound has a formula

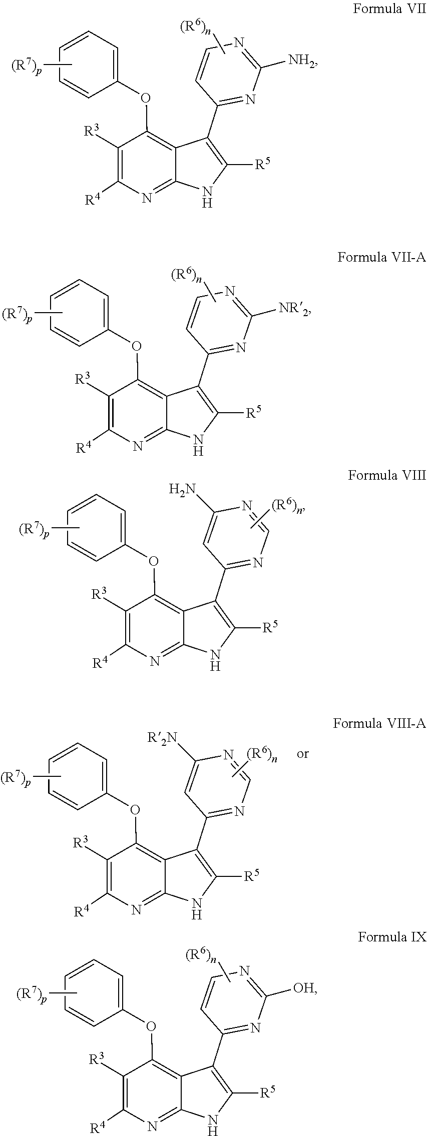

or a pharmaceutically acceptable salt thereof, where p is from 0 to 5, each R⁷ independently is amino, OH, halogen, alkyl, haloalkyl, CO₂H, CO₂R, NO₂, CN, amide, sulfonic acid, sulfonamide, hydroxyalkyl, alkoxy, or heteroaliphatic, where R is aliphatic, and each R′ independently is H, C₁₋₆alkyl or —C(O)CH₂N(C₁₋₆alkyl).

In certain embodiments, R³, R⁴ and R⁵ all are H. And in additional or alternative embodiments, n is 0.

In any embodiments, the compound may be in a free base form, or the compound may be a salt. And in some embodiments, the compound is a hydrochloride salt.

Also disclosed is a method of using the compound. In some embodiments, the method comprises administering the compound or a pharmaceutical composition thereof, to a subject in need thereof. The subject may have cancer, such as breast cancer, ovarian cancer, pancreatic cancer, or hepatocellular carcinoma. In some examples, the cancer expresses or overexpresses CDK2.

Additionally, a method of reducing or inhibiting expression or activity of CDK2 also is disclosed. The method may comprise contacting a cell with an effective amount of the compound or a pharmaceutical composition thereof.

The foregoing and other objects, features, and advantages of the invention will become more apparent from the following detailed description, which proceeds with reference to the accompanying figures.

### BRIEF DESCRIPTION OF THE DRAWINGS

FIGS. 1A-1C are a series of schematic diagrams of docking of compounds in complex with CDK2. FIG. 1A illustrates interaction of compound I-3 with CDK2. Interactions shown in green represent π-π stacking, those in pink and blue represent hydrogen bonds. Amino acid residues colored blue are the acceptor in a hydrogen bond, while amino acid residues colored pink are the donor in a hydrogen bond. FIG. 1B is a docking model of merioline-3 with CDK2. FIG. 1C shows a superposition of the predicted compound I-3 pose (in blue, at the end of MD trajectory) with the crystal pose of merioline-3 in pink (PDB code 3BHT).

FIGS. 2A-2B illustrate cell-based drug target engagement assays. FIG. 2A is a graph showing binding of the CKD2/Cyclin E, CDK1/CyclinB1, CDK9/Cyclin T1, CDK7/Cyclin H complex by I-3 (CD11) using NanoBRET target engagement intracellular kinase assay. FIG. 2B shows results of a cellular thermal shift assay (CETSA), demonstrating irreversible protein precipitation of CDK2 with increasing doses of I-3.

FIGS. 3A-3B show target protein inhibition in Basal like TNBC cells. FIG. 3A is a Western blot showing pCDK2 levels in MDA-MB-468 cells with increasing dose of I-3 (CD11). FIG. 3B is a Western blot showing Phospho-Rb levels in MDA-MB-468 cells with increasing dose of I-3 (CD11).

FIGS. 4A and 4B illustrate in vitro assessment of in vitro cell cycle analysis and Caspase 3/7 activity of C-11 in Breast Cancer Cell lines. FIG. 4A show cell cycle analysis of MDA-MB-468 cells treated for 24 hours with 1 μM I-3 (left) or 1 μM PF-06873600 (right). FIG. 4B is a graph showing Caspase Glo 3/7 results for MDA-MB-468 cells treated for 24 hours with 1-3 (CD11), PF-06873600, or staurosporine control at the indicated concentrations.

FIG. 5 is a graph showing cell viability of hepatocellular carcinoma (HCC) cell lines treated with I-3 for 72 hours, assessed by CellTiter Glo assay, with the control level of cells considered 100%.

FIG. 6 is a graph showing plasma concentration of I-3 over time after IV (1 mg/kg) or PO administration (10 mg/kg).

FIGS. 7A and 7B illustrate anti-tumor efficacy of I-3 (CD11) in MDA-MB-231 breast cancer xenograft model.

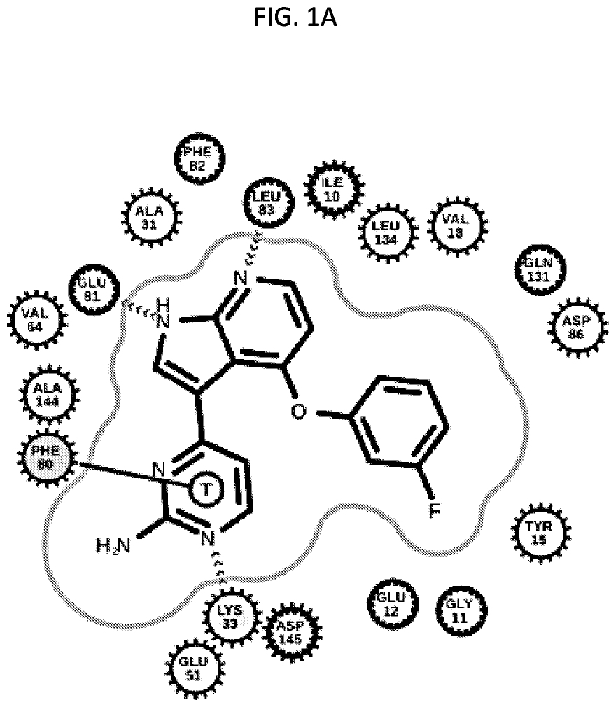

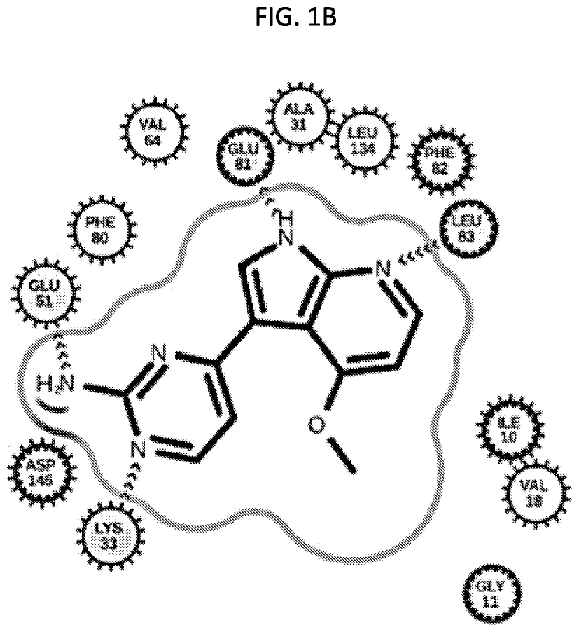

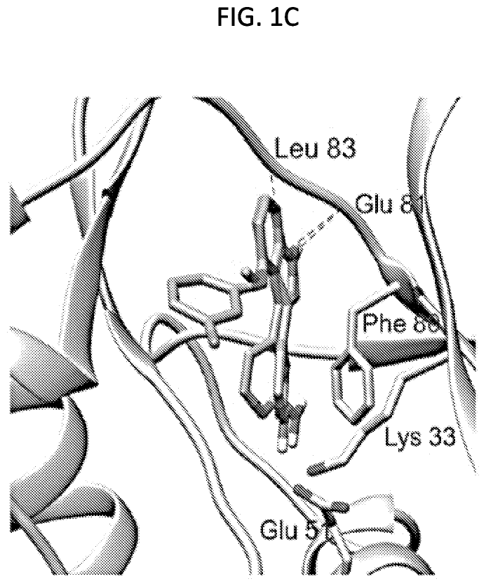

### DETAILED DESCRIPTION

### I. Terms and Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified.

Unless otherwise indicated, all numbers expressing quantities of components, molecular weights, percentages, temperatures, times, and so forth, as used in the specification or claims, are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

When chemical structures are depicted or described, unless explicitly stated otherwise, all carbons are assumed to include implicit hydrogens such that each carbon conforms to a valence of four. For example, in the structure on the left-hand side of the schematic below there are nine hydrogen atoms implied. The nine hydrogen atoms are depicted in the right-hand structure.

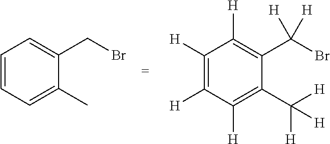

Sometimes a particular atom in a structure is described in textual formula as having a hydrogen or hydrogen atoms, for example —CH₂CH₂—. It will be understood by a person of ordinary skill in the art that the aforementioned descriptive techniques are common in the chemical arts to provide brevity and simplicity to description of organic structures.

If a group R is depicted as “floating” on a ring system, as for example in the group:

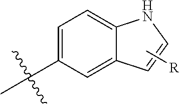

then, unless otherwise defined, a substituent R can reside on any atom of the fused bicyclic ring system, so long as a stable structure is formed that conforms to standard valence conditions as understood by a person of ordinary skill in the art. In the example depicted, the R group can reside on an atom in either the 5-membered or the 6-membered ring of the indolyl ring system, including the heteroatom by replacing the explicitly recited hydrogen, but excluding the atom carrying the bond with the “ [symbol] ” symbol and the bridging carbon atoms.

In any embodiments, any or all hydrogens present in the compound, or in a particular group or moiety within the compound, may be replaced by a deuterium or a tritium. Thus, a recitation of alkyl includes deuterated alkyl, where from one to the maximum number of hydrogens present may be replaced by deuterium. For example, ethyl may be C₂H₅ or C₂H₅ where from 1 to 5 hydrogens are replaced by deuterium, such as in C₂DₓH₅₋ₓ.

A person of ordinary skill in the art will appreciate that compounds may exhibit the phenomena of tautomerism, conformational isomerism, geometric isomerism, and/or optical isomerism. For example, certain disclosed compounds can include one or more chiral centers and/or double bonds and as a consequence can exist as stereoisomers, such as double-bond isomers (i.e., geometric isomers), enantiomers, diastereomers, and mixtures thereof, such as racemic mixtures. As another example, certain disclosed compounds can exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, a person of ordinary skill in the art will appreciate that the disclosed compounds encompass any tautomeric, conformational isomeric, optical isomeric, and/or geometric isomeric forms of the compounds described herein, as well as mixtures of these various different isomeric forms.

Any group or moiety defined herein can be connected to any other portion of a disclosed structure, such as a parent or core structure, as would be understood by a person of ordinary skill in the art, such as by considering valence rules, comparison to exemplary species, and/or considering functionality, unless the connectivity of the group or moiety to the other portion of the structure is expressly stated, or is implied by context.

“Acyl” refers to the group —C(O)R, where R is H, aliphatic, heteroaliphatic, heterocyclic or aromatic. Exemplary acyl moieties include, but are not limited to, —C(O)H, —C(O)alkyl, —C(O)C₁-C₆alkyl, —C(O)C₁-C₆haloalkyl, —C(O)cycloalkyl, —C(O)alkenyl, —C(O)cycloalkenyl, —C(O)aryl, —C(O)heteroaryl, or —C(O)heterocyclyl. Specific examples include —C(O)H, —C(O)Me, —C(O)Et, or —C(O)cyclopropyl.

“Aliphatic” refers to a substantially hydrocarbon-based group or moiety. An aliphatic group or moiety can be acyclic, including alkyl, alkenyl, or alkynyl groups, cyclic versions thereof, such as cycloaliphatic groups or moieties including cycloalkyl, cycloalkenyl or cycloalkynyl, and further including straight- and branched-chain arrangements, and fused and bridged arrangements with respect to the cyclic versions, and all stereo and position isomers as well. Unless expressly stated otherwise, an aliphatic group contains from one to twenty-five carbon atoms (C₁₋₂₅); for example, from one to fifteen (C₁₋₁₅), from one to ten (C₁₋₁₀), from one to six (C₁₋₆), from one to four carbon atoms (C₁₋₄) or two to twenty two (C₂₋₂₂) or 6 to 18 (C₆₋₁₈) for a saturated acyclic aliphatic group or moiety, from two to twenty-five carbon atoms (C₂₋₂₅); for example, from two to fifteen (C₂₋₁₅), from two to ten (C₂₋₁₀), from two to six (C₂₋₆), or from two to four carbon atoms (C₂₋₄) for an unsaturated acyclic aliphatic group or moiety, or from three to fifteen (C₃₋₁₅) from three to ten (C₃₋₁₀), from three to six (C₃₋₆), or from three to four (C₃₋₄) carbon atoms for a cycloaliphatic group or moiety. An aliphatic group may be substituted or unsubstituted, unless expressly referred to as an “unsubstituted aliphatic” or a “substituted aliphatic.” An aliphatic group can be substituted with one or more substituents (up to two substituents for each methylene carbon in an aliphatic chain, or up to one substituent for each carbon of a —C═C— double bond in an aliphatic chain, or up to one substituent for a carbon of a terminal methine group). Substituents on an aliphatic group or moiety may be any substituents understood by a person of ordinary skill in the art to be compatible with the synthesis of the oleofuran compounds. Exemplary substituents include, but are not limited to, hydroxyl, amine, carbonyl (C═O), aldehyde, or aliphatic, such as alkyl, alkenyl, alkynyl, and straight chain, cyclic and branched versions thereof.

“Alkoxy” refers to a —O-alkyl group.

“Alkyl” refers to a saturated aliphatic hydrocarbyl group having from 1 to 25 (C₁₋₂₅) or more carbon atoms, such as from 1 to 10 (C₁₋₁₀) carbon atoms, from 1 to 6 (C₁₋₆) carbon atoms, or from 2 to 22 (C₂₋₂₂) carbon atoms or from 6 to 18 (C₆₋₁₈) carbon atoms. An alkyl moiety may be substituted or unsubstituted. This term includes, by way of example, linear and branched hydrocarbyl groups such as methyl (CH₃), ethyl (—CH₂CH₃), n-propyl (—CH₂CH₂CH₃), isopropyl (—CH(CH₃)₂), n-butyl (—CH₂CH₂CH₂CH₃), isobutyl (—CH₂CH₂(CH₃)₂), sec-butyl (—CH(CH₃)(CH₂CH₃), t-butyl (—C(CH₃)₃), n-pentyl (—CH₂CH₂CH₂CH₂CH₃), neopentyl (—CH₂C(CH₃)₃), hexyl (C₆H₁₃), heptyl (C₇H₁₅), octyl (C₈H₁₇), decyl (C₁₀H₂₁), dodecyl (C₁₂H₂₅), tetradecyl (C₁₄H₂₉), hexadecyl (C₁₆H₃₃), octadecyl (C₁₈H₃₇) or eicosanyl (C₂₀H₄₁).

“Alkylamino” refers to a -alkyl-amino moiety, where the alkyl and amino moieties are as defined herein.

“Amide” refers to a —C(O)amino moiety.

“Amino” refers to a —N(R)R′ moiety where R and R′ are independently H, aliphatic, such as alkyl, alkenyl or alkynyl, or R and R′ together with the nitrogen to which they are attached for a 5- to 7-membered heterocyclic ring, optionally containing one, two or three further heteroatoms selected from O, N or S, and/or optionally substituted with one, two or three aliphatic groups, such as alkyl groups.

“Aromatic” refers to a cyclic, conjugated group or moiety of, unless specified otherwise, from 5 to 15 ring atoms having a single ring (e.g., phenyl, pyridinyl, or pyrazolyl) or multiple condensed rings in which at least one ring is aromatic (e.g., naphthyl, indolyl, or pyrazolopyridinyl), that is at least one ring, and optionally multiple condensed rings, have a continuous, delocalized π-electron system. Typically, the number of out of plane π-electrons corresponds to the Huckel rule (4n+2). The point of attachment to the parent structure typically is through an aromatic portion of the condensed ring system. For example,

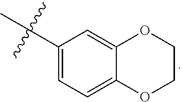 [SMILES: *c1ccc2c(c1)OCCO2]

However, in certain examples, context or express disclosure may indicate that the point of attachment is through a non-aromatic portion of the condensed ring system. For example,

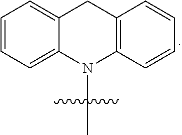 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g. S, O, N, P, or Si), such as in a heteroaryl group or moiety. Unless otherwise stated, an aromatic group may be substituted or unsubstituted.

“Aryl” refers to an aromatic carbocyclic group of, unless specified otherwise, from 6 to 15 carbon atoms having a single ring (e.g., phenyl) or multiple condensed rings in which at least one ring is aromatic (e.g., 1,2,3,4-tetrahydroquinoline, benzodioxole, and the like). If any aromatic ring portion contains a heteroatom, the group is heteroaryl and not aryl. Aryl groups may be, for example, monocyclic, bicyclic, tricyclic or tetracyclic. Unless otherwise stated, an aryl group may be substituted or unsubstituted.

“Breast cancer” refers to a malignant neoplasm that arises in or from breast tissue (such as a ductal carcinoma). Breast cancers are frequently classified as luminal A (ER positive and/or PR positive, ErbB2 negative, and low Ki67), luminal B (ER positive and/or PR positive and ErbB2 positive, or ErbB2 negative with high Ki67), basal-like or triple-negative (ER negative, PR negative, ErbB2 negative, cytokeratin 5/6 positive and/or HER1 positive), or ErbB2 positive (ER negative, PR negative, ErbB2 positive). However, breast cancers may be heterogeneous both between individuals and at the cellular level within a tumor, and may not always fit within the classification scheme.

“Triple negative breast cancer” (TNBC) is a subtype of breast cancer characterized by lack of expression of estrogen and progesterone receptors (ER/PR) and lack expression of (or lack overexpression of) human epidermal growth factor receptor-2 (referred to as HER2 or ErbB2) in the tumor cells. In some examples, TNBC is invasive ductal carcinoma or ductal carcinoma in situ. In other examples, TNBC is basal-like breast cancer. The pathological features of TNBC may include lymphocytic infiltrate, pushing borders, high mitotic rate (>19/10 HPF), central necrosis, medullary features, and metaplastic elements (e.g., squamous cells and spindle cells).

“Cancer” refers to a malignant neoplasm that has undergone anaplasia with loss of differentiation, increased rate of growth, invasion of surrounding tissue, and is capable of metastasis. As used herein, cancer includes both solid tumors and hematological malignancies. Residual cancer is cancer that remains in a subject after any form of treatment is given to the subject to reduce or eradicate cancer. Metastatic cancer is a cancer at one or more sites in the body other than the original site of the cancer from which the metastatic cancer is derived. Local recurrence is a reoccurrence of the cancer at or near the same site as the original cancer, for example, in the same tissue as the original cancer.

“Carboxyl ester” or “carboxy ester” refers to the group —C(O)OR, where R is aliphatic, heteroaliphatic, heterocyclic, or aromatic, including both aryl and heteroaryl.

“Carrier or Vehicle” refers to an excipient that serves as a component capable of delivering a compound described herein. In some embodiments, a carrier can be a suspension aid, solubilizing aid, or aerosolization aid. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In some examples, the pharmaceutically acceptable carrier may be sterile to be suitable for administration to a subject (for example, by parenteral, intramuscular, or subcutaneous injection). In addition to biologically-neutral carriers, pharmaceutical formulations to be administered can contain minor amounts of non-toxic auxiliary substances, such as wetting or emulsifying agents, preservatives, and pH buffering agents and the like, for example sodium acetate or sorbitan monolaurate.

“Cyclin-Dependent Kinase (CDK)” refers to a family of serine/threonine protein kinases that interact with cyclins and are involved in cell cycle regulation. Major CDKs in humans include CDK1, CDK2, CDK4, and CDK6. Abnormal regulation of the CDK4- and CDK6-cyclin D-INK4-retinoblastoma protein (Rb) signaling pathway is among the most common aberrations found in many human cancers.

“Halo,” “halide” or “halogen” refers to fluoro, chloro, bromo or iodo.

“Heteroaliphatic” refers to an aliphatic compound or group having at least one heteroatom and at least one carbon atom, i.e., one or more carbon atoms from an aliphatic compound or group comprising at least two carbon atoms, has been replaced with an atom having at least one lone pair of electrons, typically nitrogen, oxygen, phosphorus, silicon, or sulfur. Heteroaliphatic compounds or groups may be substituted or unsubstituted, branched or unbranched, chiral or achiral, and/or acyclic or cyclic, such as a heterocycloaliphatic group.

“Haloalkyl” refers to an alkyl moiety substituted with one or more halogens. Exemplary haloalkyl moieties include —CH₂F, —CHF₂ and —CF₃.

“Heteroaryl” refers to an aromatic group or moiety of, unless specified otherwise, from 5 to 15 ring atoms comprising at least one carbon atom and at least one heteroatom, such as N, S, O, P, or Si, typically N, O or S. A heteroaryl group or moiety may comprise a single ring (e.g., pyridinyl, pyrimidinyl or pyrazolyl) or multiple condensed rings (e.g., indolyl, benzopyrazolyl, or pyrazolopyridinyl). Heteroaryl groups or moiety may be, for example, monocyclic, fused, such as bicyclic, tricyclic or tetracyclic, or spriocyclic. Unless otherwise stated, a heteroaryl group or moiety may be substituted or unsubstituted.

“Heterocyclyl” refers to both aromatic and non-aromatic ring systems unless otherwise specified, and more specifically refer to a stable three- to fifteen-membered ring moiety comprising at least one carbon atom, and typically plural carbon atoms, and at least one, such as from one to five, heteroatoms. Typical heteroatoms include nitrogen, oxygen, sulfur, or a combination thereof. The heterocyclyl moiety may be a monocyclic moiety, or may comprise multiple rings, such as in a bicyclic or tricyclic ring system, provided that at least one of the rings contains a heteroatom. Such a multiple ring moiety can include fused or bridged ring systems as well as spirocyclic systems; and any nitrogen, phosphorus, carbon, silicon or sulfur atoms in the heterocyclyl moiety can be optionally oxidized to various oxidation states. For convenience, nitrogens, particularly, but not exclusively, those defined as annular aromatic nitrogens, are meant to include their corresponding N-oxide form, although not explicitly defined as such in a particular example. Thus, for a compound having, for example, a pyridinyl ring, the corresponding pyridinyl-N-oxide is included as another compound of the invention, unless expressly excluded or excluded by context. In addition, annular nitrogen atoms can be optionally quaternized. Heterocyclyl groups includes aromatic, heteroaryl moieties, and non-aromatic heterocycloaliphatic moieties, which are heterocyclyl rings that are partially or fully saturated. Examples of heterocyclyl groups include, but are not limited to, pyridinyl, pyrimidinyl, pyrazinyl, triazinyl, indolyl, pyrazolyl, imidazolyl, furanyl, thiophenyl, thiazolyl, oxazolyl, isoxazolyl, isothiazolyl, oxadiazolyl, tetrazolyl, quinolyl, indolyl, benzodioxanyl, benzofuranyl, benzothiophenyl, pyrazolopyridinyl, morpholinyl, piperadinyl, piperazinyl, pyrrolidinyl, homopiperadinyl, imidazolidinyl, or pyrazolidinyl.

“Hydroxy” refers to a —OH moiety.

“Pharmaceutically acceptable excipient” refers to a substantially physiologically inert substance that is used as an additive in a pharmaceutical composition. As used herein, an excipient may be incorporated within particles of a pharmaceutical composition, or it may be physically mixed with particles of a pharmaceutical composition. An excipient can be used, for example, as a carrier, flavoring, thickener, diluent, buffer, preservative, or surface active agent and/or to modify properties of a pharmaceutical composition. Examples of excipients include, but are not limited, to polyvinylpyrrolidone (PVP), tocopheryl polyethylene glycol 1000 succinate (also known as vitamin E TPGS, or TPGS), dipalmitoyl phosphatidyl choline (DPPC), trehalose, sodium bicarbonate, glycine, sodium citrate, and lactose.

“Pharmaceutically acceptable salt” refers to a biologically compatible salt of a compound that can be used as a drug, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. Pharmaceutically acceptable acid addition salts are those salts that retain the biological effectiveness of the free bases while formed by acid partners that are not biologically or otherwise undesirable, e.g., inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, benzene sulfonic acid (besylate), cinnamic acid, mandelic acid, methanesulfonic acid, ethanesulfonic acid, p-toluenesulfonic acid, salicylic acid and the like. Pharmaceutically acceptable base addition salts include those derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19, which is incorporated herein by reference.)

“Subject” refers to mammals and other animals, such as humans, companion animals (e.g., dogs, cats, rabbits, etc.), utility animals, and feed animals; thus, disclosed methods are applicable to both human therapy and veterinary applications.

“Sulfonamide” refers to the group —SO₂amino, where amino is as defined herein.

“Sulfonic acid” refers to the group —SO₂OH.

“Therapeutically Effective Amount” refers to an amount of a compound sufficient to treat a specified disorder or disease, or to ameliorate or eradicate one or more of its symptoms and/or to inhibit occurrence or recurrence of the disease or disorder. The amount of a compound which constitutes a “therapeutically effective amount” will vary depending on the compound, the disease state and its severity, the age and condition of the patient to be treated, and the like. The therapeutically effective amount can be determined by a person of ordinary skill in the art.

“Treating, Treatment, and Therapy” refers to any success or indicia of success in the attenuation or amelioration of an injury, pathology or condition, including any objective or subjective parameter such as abatement, remission, diminishing of symptoms or making the condition more tolerable to the patient, slowing in the rate of disease progression, or improving a subject's physical or mental well-being. The treatment may be assessed by objective or subjective parameters; including the results of a physical examination, neurological examination, and/or psychiatric evaluation.

As used herein, the terms “disease” and “condition” can be used interchangeably or can be different in that the particular malady or condition may not have a known causative agent (so that etiology has not yet been determined) and it is therefore not yet recognized as a disease but only as an undesirable condition or syndrome, where a more or less specific set of symptoms have been identified by clinicians.

### II. Overview

Triple-negative breast cancer (TNBC) is a heterogeneous group of tumors cancers defined by the absence of estrogen receptor (ER), progesterone receptor (PR), and overexpression of human epidermal growth factor receptor 2 (HER2) gene. TNBC comprises 10-20% of invasive breast cancers and has been found to be associated with higher grade and mitotic index, younger age and African-American race. Due to lack of drug-targetable receptors, treatment options are more limited to surgery with or without chemotherapy and adjuvant radiotherapy. In addition to fewer treatment options, survival after metastatic relapse in TNBC is shorter as compared to other breast cancer subtypes, and treatment response rates are poor and lack durability. Abnormalities of the cell-cycle are a pervasive finding in human breast cancer and other malignancies. Cyclin-dependent kinases (CDKs) are the families of protein kinases that play important roles in regulating the cell cycle. CDK 4/6 inhibitors in combination with hormonal therapy are FDA-approved for the first- or second-line treatment of patients with ER⁺ HER2⁻ advanced or metastatic breast cancer. However, not all patients respond to current CDK inhibitors.

Previous work established the cyclinD-CDK4/6 complex is implicated in retinoblastoma phosphorylation, which led to the approval of the CDK4/6 inhibitors Palbociclib (Pfizer), Ribociclib (Novaratis), and Abemaciclib (Eli Lilly) for the treatment of intact retinoblastoma breast tumors in combination with endocrine therapy. However, these CDK4/6 inhibitors perform poorly in low retinoblastoma expressing triple negative breast cancer (TNBC) known as the basal like breast cancer subtype. Specifically, the retinoblastoma tumor suppressor gene (RB) is lost in an estimated 30% of TNBCs. Typically, the RB tumor suppressor plays a key role in cell cycle regulation. However, if mutated, the RB tumor suppressor cannot inhibit cell cycle progression, contributing to tumorigenesis. All existing CDK4/6 inhibitors used in the treatment of hormone-receptor positive and some subtypes of TNBC rely on an intact RB for success. However, there are current no effective selective therapies for RB loss in TNBC.

While TNBC (e.g., basal-like breast cancer (BLBC)) is characterized by low expression of the RB and Cyclin D1 genes, it also highly expresses the E2F3 and Cyclin E genes. Cyclin E1, a regulator of CDK2, is present in higher copy numbers in BLBC than other molecular subtypes, and its expression correlates with poor survival in breast cancer. Similarly, in hepatocellular carcinoma, upregulation of CDK2 and its partner, cyclin E1, are a frequent finding, and data from the Human Protein Atlas indicate that high expression of CDK2 is associated with an inferior survival. These studies suggest a specific role of CDK2 and it is, therefore, a target for inhibition, and its use across all stages would represent a paradigm shift in the management of TNBC and HCC. Notably, there are currently no CDK2-specific inhibitors available, although some pan-CDK inhibitors, such as PF-06873600 (NCT03519178) and CYC065 (NCT02552953), have been shown to target CDK2 with a higher affinity and have progressed to Phase I clinical trials. There is still a need to develop novel CDK2 inhibitors with potential in treating cancers, including those with RB loss.

### III. Compounds

Disclosed herein are compounds that may be useful in the treatment of cancer. In some examples, the compounds inhibit activity of one or more CDKs, such as CDK2. In some embodiments, the compounds have a structure according to Formula I

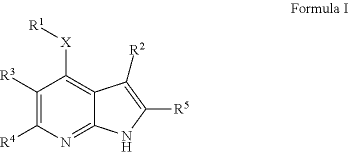

or a pharmaceutically acceptable salt thereof.

With respect to Formula I, R¹ is aromatic, such as aryl or heteroaryl, heterocycloaliphatic, or cycloalkyl. R¹ may be a single ring or a fused ring, such as a phenyl, naphthyl, pyridinyl, pyrimidinyl, pyrazinyl, triazinyl, indolyl, pyrazolyl, imidazolyl, furanyl, thiophenyl, thiazolyl, oxazolyl, isoxazolyl, isothiazolyl, oxadiazolyl, tetrazolyl, quinolyl, indolyl, benzodioxanyl, benzofuranyl, benzothiophenyl, pyrazolopyridinyl, piperidinyl, cyclopropyl, cyclobutyl, cyclopentyl, cyclohexyl, bicyclo[1.1.1]pentanyl, bicyclo[2.1.1]hexanyl, bicyclo[2.2.1]heptanyl, or bicyclo[2.2.2]octanyl.

In some embodiments, R¹ is unsubstituted, but in other embodiments, R¹ is substituted with one or more substituents, such as with 1, 2, 3, 4, 5 or more substituents, as permitted by chemical valency rules of the R¹ moiety. Suitable substituents for R¹ include, but are not limited to, halogen, such as F, Cl, Br or I; alkyl, such as C₁₋₆alkyl, for example, methyl, ethyl, propyl, isopropyl; cycloalkyl, such as C₃₋₆alkyl, for example, cyclopropyl, cyclopentyl, or cyclohexyl; Haloalkyl, such as C₁₋₆haloalkyl, for example, CF₃, CHF₂, CH₂F, CH₂CF₃, C₂F₅; CO₂H, CO₂R where R is C₁₋₆alkyl or C₃₋₆cycloalkyl; NO₂; CN; OH; amino, such as NH₂, NMe₂, NEt₂ or a cyclic amino such as piperazine, piperidine, or morpholine; amide, such as C(O)NH₂ or C(O)NMe₂; alkylamino, such as CH₂NH₂, CH₂NMe₂, —CH₂piperazine, —CH₂piperidine, or —CH₂morpholine; amide, such as CONH₂, CONMe₂; sulfonic acid; sulfonamide, such as SO₂NH₂, SO₂NMe₂; hydroxyalkyl, such as CH₂OH, or CH₂CH₂OH; alkoxy, such as methoxy or ethoxy; heteroaliphatic, such as —CH₂CH₂OCH₂CH₃; or a combination thereof.

X is O, S, SO₂, CH₂, NH, NMe, or a bond.

R² is heteroaryl, typically a 5- or 6-membered nitrogen-containing heteroaryl, for example, pyrimidinyl, pyridinyl, pyrazinyl, triazinyl, pyrrolyl, pyrazolyl, imidazolyl, triazolyl, oxadiazolyl, oxazolyl, isoxazolyl, thiazolyl, thiadiazolyl

R² may be unsubstituted or substituted. In some embodiments, R² is substituted with amine and/or hydroxy, such as NR′₂ and/or OH, where each R′ independently is H, C₁₋₆alkyl or —C(O)CH₂N(C₁₋₆alkyl)₂, and may be further substituted with additionally amino and/or OH groups, and/or halogen, alkyl, haloalkyl, CO₂H, CO₂R where R is alkyl, NO₂, CN, amide, sulfonic acid, sulfonamide, hydroxyalkyl, alkoxy, heteroaliphatic, or a combination thereof. In some embodiments, R² is substituted with at least NH₂, OH, or —NHC(O)CH₂N(CH₃)₂.

Each of R³, R⁴ and R⁵ independently is H or aliphatic, such as C₁₋₆alkyl.

In some embodiments, R² is pyrimidinyl substituted with at least OH, amino, such as NH₂ or —NHC(O)CH₂N(CH₃)₂. In some embodiments, the compound has a structure according to one of Formulas II, II-A, III, II-A, or IV

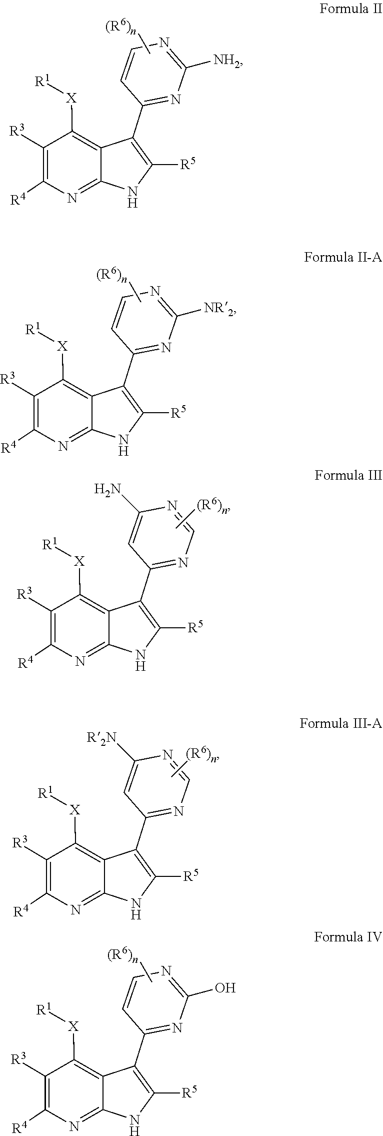

With respect to Formulas II, II-A, III, III-A, and IV, R¹, R³, R⁴ and R⁵ are as defined above for Formula I, n is 0, 1 or 2, and each R⁶ independently is amino, OH, halogen, alkyl, haloalkyl, CO₂H, CO₂R, NO₂, CN, amide, sulfonic acid, sulfonamide, hydroxyalkyl, alkoxy, or heteroaliphatic, where R is aliphatic, such as alkyl. And if present, each R′ independently is H or —C(O)CH₂N(CH₃)₂.

In some embodiments, R² is pyridinyl, typically substituted with amino or OH. In some embodiments, the compound has a structure according to one of Formulas V, V-A or VI

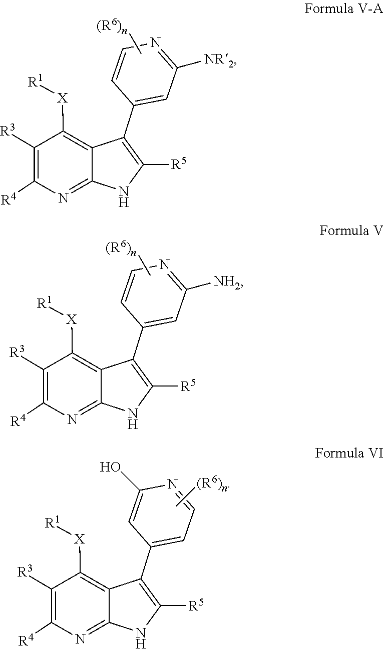

With respect to Formulas V or VI, R¹, R³, R⁴ and R⁵ are as defined above for Formula I, n is from 0 to 3, R′ is as defined for Formulas II and III, and R⁶ is as defined for Formulas II, II-A, III, III-A, and IV.

Additionally with respect to Formulas I-VI:
in some embodiments, X is O;
in some embodiments, R³, R⁴ and R⁵ are H;
in some embodiments, n is O;
in some embodiments, R¹ is phenyl, pyridinyl, pyrimidinyl, quinolinyl, imidazolyl, pyrazinyl, or furanyl;
or a combination thereof.

In certain embodiments, the compound has a structure according to one of Formulas VII, VII-A, VIII, VIII-A or IX

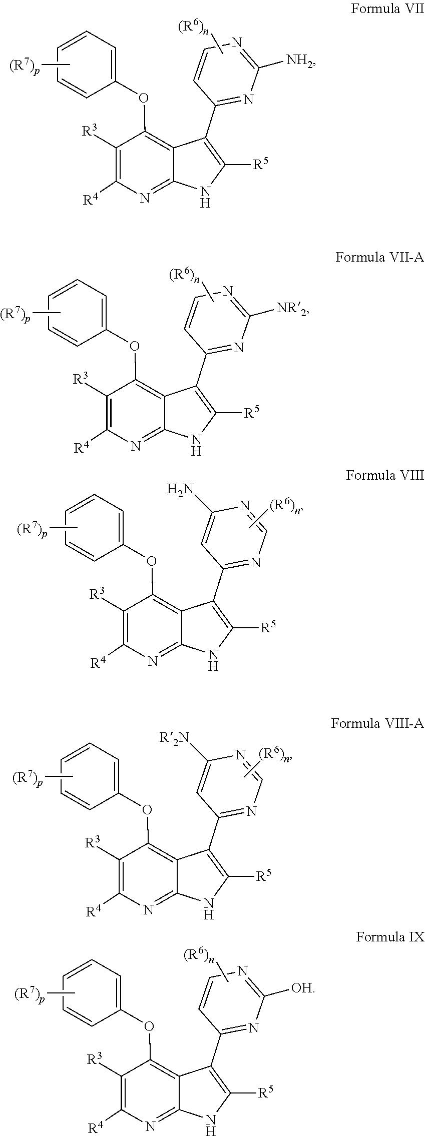

With respect to Formulas VII, VIII and IX, R³, R⁴ and R⁵ are as defined above for Formulas I-VI, R⁶, R′ and n are as defined above for Formulas II-VI, p is from 0 to 5, such as 0, 1, 2, 3, 4, or 5, and each R⁷ independently is amino, OH, halogen, alkyl, haloalkyl, CO₂H, CO₂R, NO₂, CN, amide, sulfonic acid, sulfonamide, hydroxyalkyl, alkoxy, or heteroaliphatic, where R is as previously defined for R⁶.

In some embodiments, the compound is in a free base from, i.e., not in a salt form. But in other embodiments, the compound is a salt, such as a hydrochloride salt.

Exemplary compounds according to any one of Formulas I-IX include:

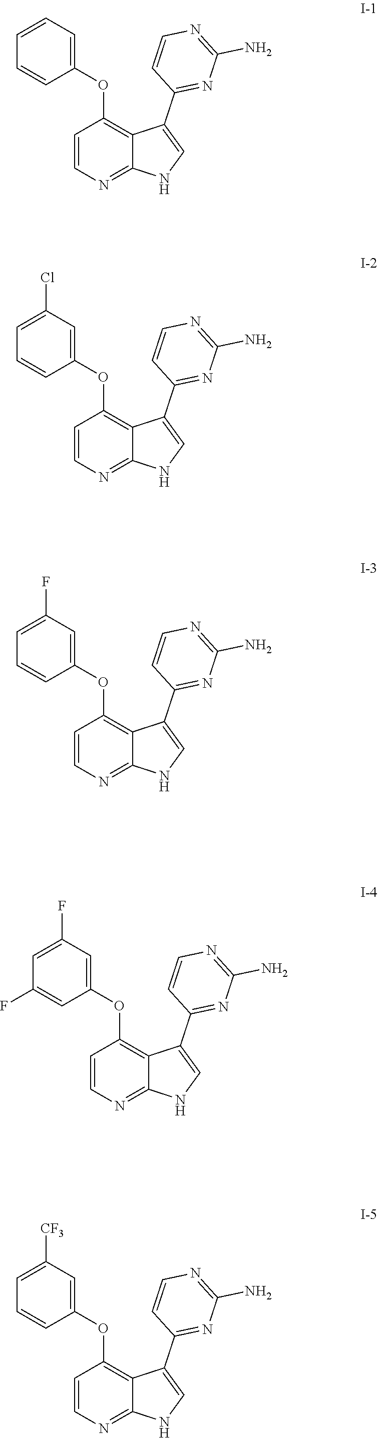 [SMILES: Nc1nccc(-c2c[nH]c3nccc(Oc4ccccc4)c23)n1 ; Nc1nccc(-c2c[nH]c3nccc(Oc4cccc(Cl)c4)c23)n1 ; Nc1nccc(-c2c[nH]c3nccc(Oc4cccc(F)c4)c23)n1 ; Nc1nccc(-c2c[nH]c3nccc(Oc4cc(F)cc(F)c4)c23)n1 ; Nc1nccc(-c2c[nH]c3nccc(Oc4cccc(C(F)(F)F)c4)c23)n1]

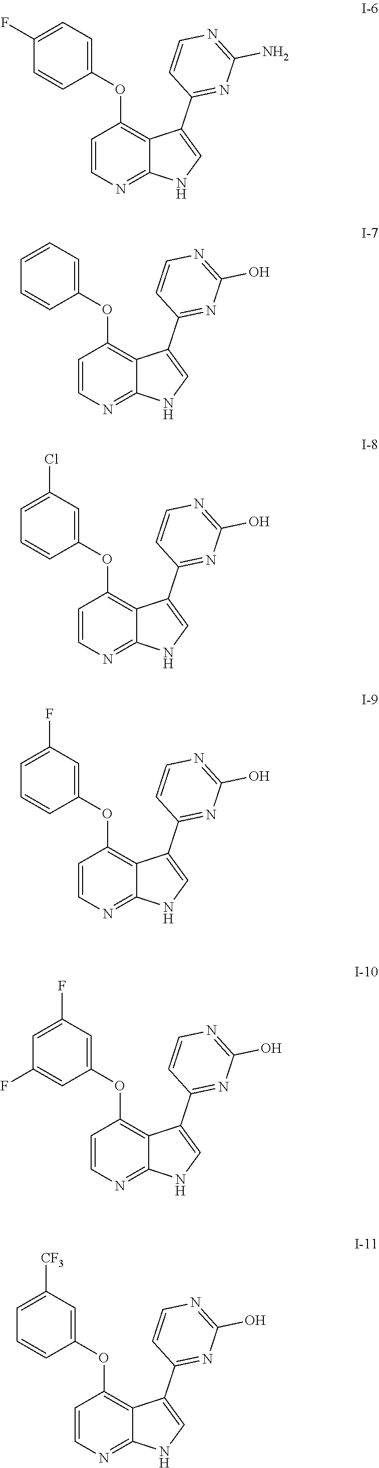 [SMILES: Nc1nccc(-c2c[nH]c3nccc(Oc4ccc(F)cc4)c23)n1 ; Oc1nccc(-c2c[nH]c3nccc(Oc4ccccc4)c23)n1 ; Oc1nccc(-c2c[nH]c3nccc(Oc4cccc(Cl)c4)c23)n1 ; Oc1nccc(-c2c[nH]c3nccc(Oc4cccc(F)c4)c23)n1 ; Oc1nccc(-c2c[nH]c3nccc(Oc4cc(F)cc(F)c4)c23)n1 ; Oc1nccc(-c2c[nH]c3nccc(Oc4cccc(C(F)(F)F)c4)c23)n1]

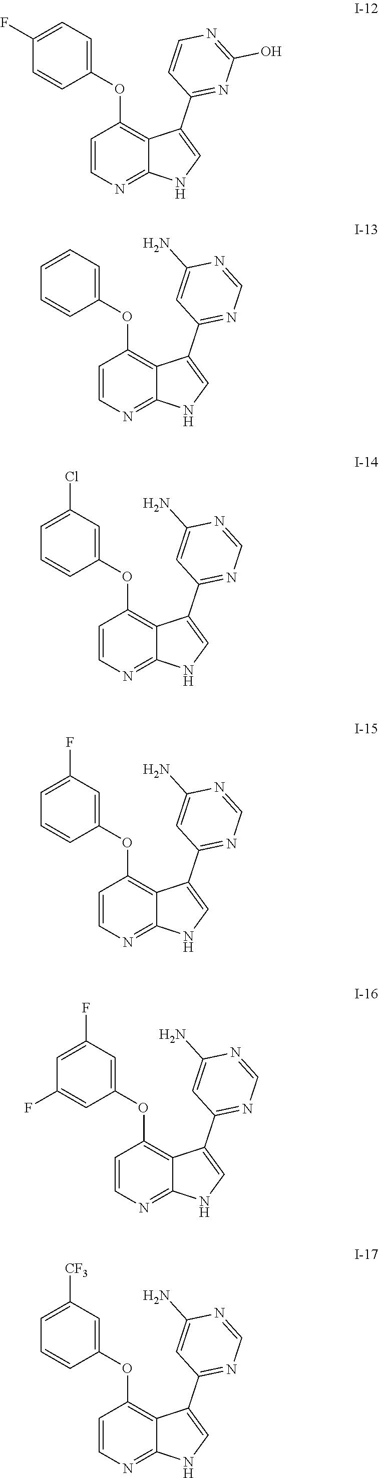 [SMILES: Oc1nccc(-c2c[nH]c3nccc(Oc4ccc(F)cc4)c23)n1 ; Nc1cc(-c2c[nH]c3nccc(Oc4ccccc4)c23)ncn1 ; Nc1cc(-c2c[nH]c3nccc(Oc4cccc(Cl)c4)c23)ncn1 ; Nc1cc(-c2c[nH]c3nccc(Oc4cccc(F)c4)c23)ncn1 ; Nc1cc(-c2c[nH]c3nccc(Oc4cc(F)cc(F)c4)c23)ncn1 ; Nc1cc(-c2c[nH]c3nccc(Oc4cccc(C(F)(F)F)c4)c23)ncn1]

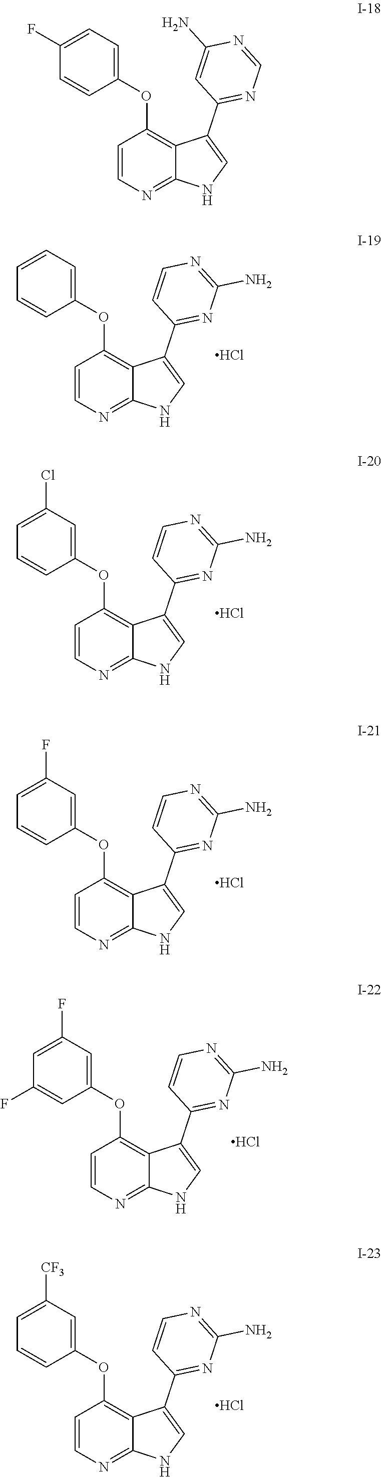

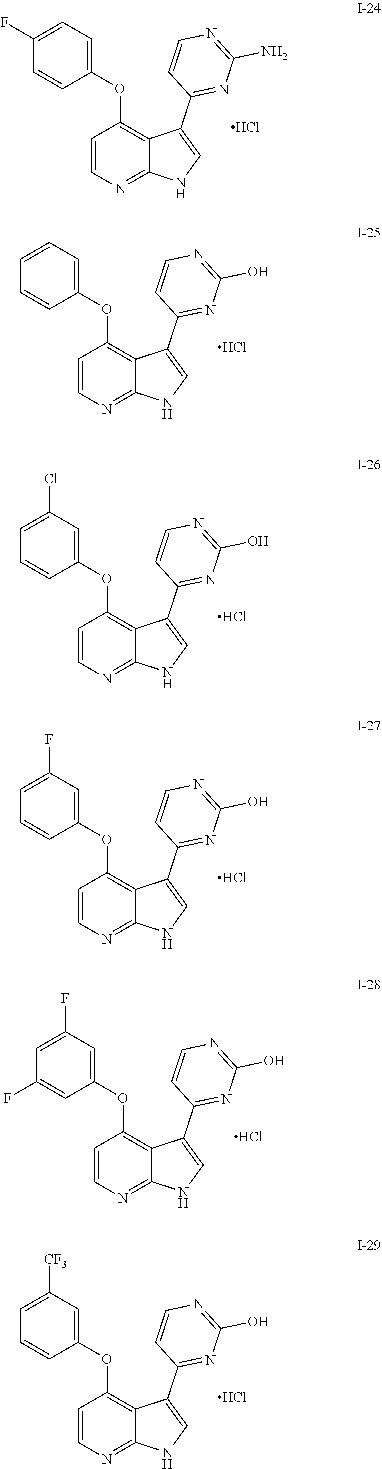

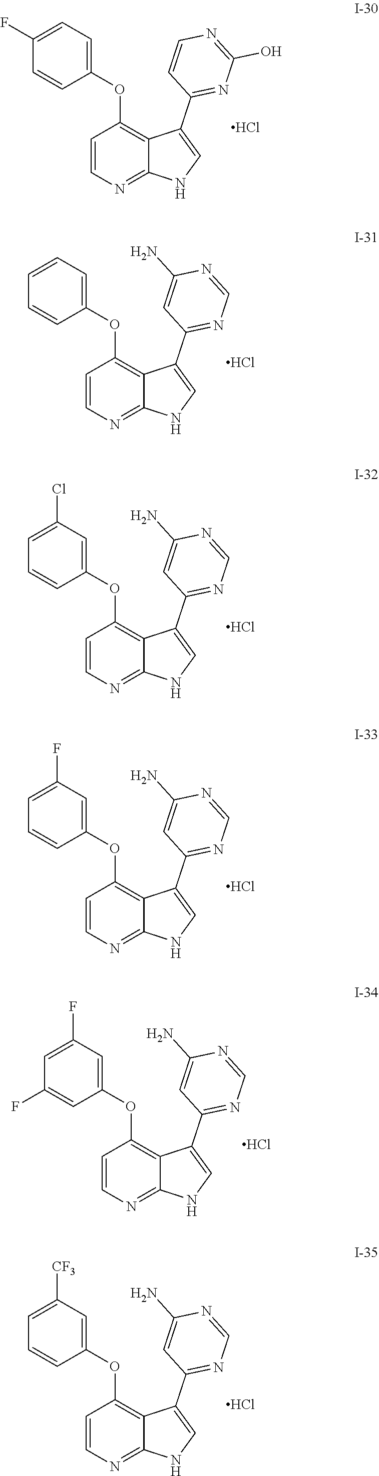

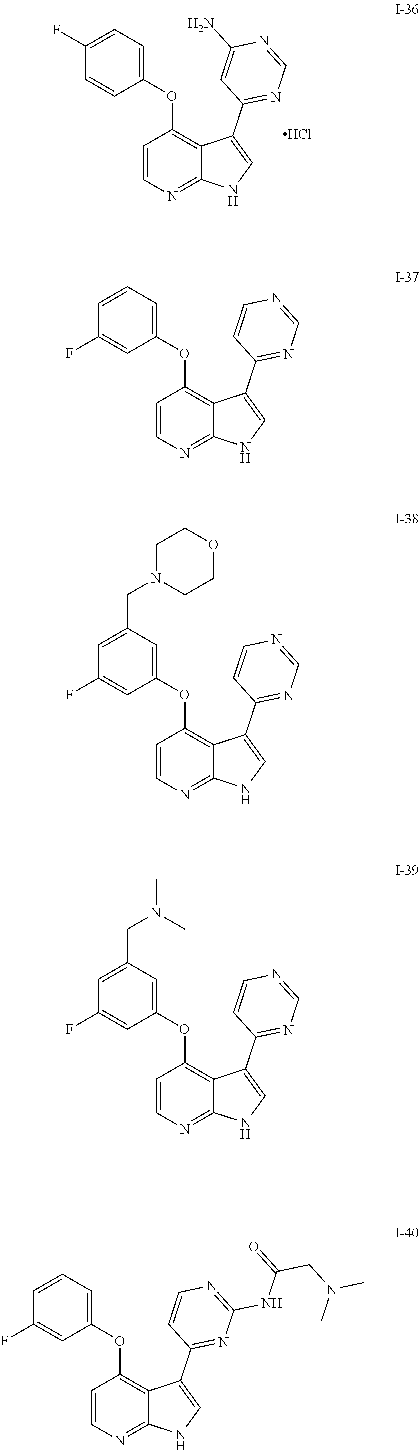 [SMILES: Nc1cc(-c2c[nH]c3nccc(Oc4ccc(F)cc4)c23)ncn1 ; Cl ; Fc1cccc(Oc2ccnc3[nH]cc(-c4ccncn4)c23)c1 ; Fc1cc(CN2CCOCC2)cc(Oc2ccnc3[nH]cc(-c4ccncn4)c23)c1 ; CN(C)Cc1cc(F)cc(Oc2ccnc3[nH]cc(-c4ccncn4)c23)c1 ; CN(C)CC(=O)Nc1nccc(-c2c[nH]c3nccc(Oc4cccc(F)c4)c23)n1]

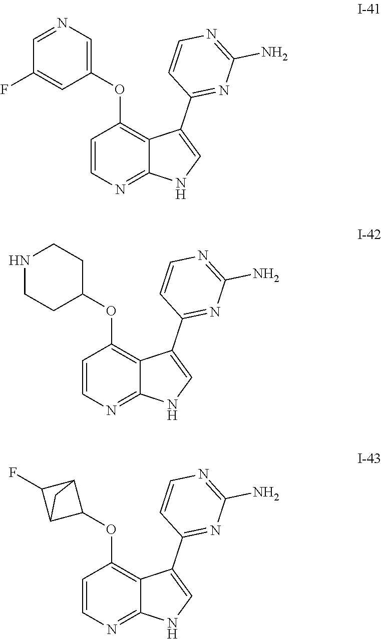 [SMILES: Nc1nccc(-c2c[nH]c3nccc(Oc4cncc(F)c4)c23)n1 ; Nc1nccc(-c2c[nH]c3nccc(OC4CCNCC4)c23)n1 ; Nc1nccc(-c2c[nH]c3nccc(OC4C5CC4C5F)c23)n1]

Other exemplary compounds according to any one of Formulas I-IX may include:
I-1: 4-(4-phenoxy-1H-pyrrolo[2,3-b]pyridin-3-yl)pyrimidin-2-amine;
I-2: 4-(4-(3-chlorophenoxy)-1H-pyrrolo[2,3-b]pyridin-3-yl)pyrimidin-2-amine;
I-3: 4-(4-(3-fluorophenoxy)-1H-pyrrolo[2,3-b]pyridin-3-yl)pyrimidin-2-amine;
I-4: 4-(4-(3,5-difluorophenoxy)-1H-pyrrolo[2,3-b]pyridin-3-yl)pyrimidin-2-amine;
I-5: 4-(4-(3-(trifluoromethyl)phenoxy)-1H-pyrrolo[2,3-b]pyridin-3-yl)pyrimidin-2-amine;
I-6: 4-(4-(4-fluorophenoxy)-1H-pyrrolo[2,3-b]pyridin-3-yl)pyrimidin-2-amine;
I-7: 4-(4-phenoxy-1H-pyrrolo[2,3-b]pyridin-3-yl)pyrimidin-2-ol;
I-8: 4-(4-(3-chlorophenoxy)-1H-pyrrolo[2,3-b]pyridin-3-yl)pyrimidin-2-ol;
I-9: 4-(4-(3-fluorophenoxy)-1H-pyrrolo[2,3-b]pyridin-3-yl)pyrimidin-2-ol;
I-10: 4-(4-(3,5-difluorophenoxy)-1H-pyrrolo[2,3-b]pyridin-3-yl)pyrimidin-2-ol;
I-11: 4-(4-(3-(trifluoromethyl)phenoxy)-1H-pyrrolo[2,3-b]pyridin-3-yl)pyrimidin-2-ol;
I-12: 4-(4-(4-fluorophenoxy)-1H-pyrrolo[2,3-b]pyridin-3-yl)pyrimidin-2-ol;
I-13: 6-(4-phenoxy-1H-pyrrolo[2,3-b]pyridin-3-yl)pyrimidin-4-amine;
I-14: 6-(4-(3-chlorophenoxy)-1H-pyrrolo[2,3-b]pyridin-3-yl)pyrimidin-4-amine;
I-15: 6-(4-(3-fluorophenoxy)-1H-pyrrolo[2,3-b]pyridin-3-yl)pyrimidin-4-amine;
I-16: 6-(4-(3,5-difluorophenoxy)-1H-pyrrolo[2,3-b]pyridin-3-yl)pyrimidin-4-amine;
I-17: 6-(4-(3-(trifluoromethyl)phenoxy)-1H-pyrrolo[2,3-b]pyridin-3-yl)pyrimidin-4-amine;
1-18: 6-(4-(4-fluorophenoxy)-1H-pyrrolo[2,3-b]pyridin-3-yl)pyrimidin-4-amine;
I-19: 4-(4-phenoxy-1H-pyrrolo[2,3-b]pyridin-3-yl)pyrimidin-2-amine hydrochloride;
I-20: 4-(4-(3-chlorophenoxy)-1H-pyrrolo[2,3-b]pyridin-3-yl)pyrimidin-2-amine hydrochloride;
I-21: 4-(4-(3-fluorophenoxy)-1H-pyrrolo[2,3-b]pyridin-3-yl)pyrimidin-2-amine hydrochloride;
I-22: 4-(4-(3,5-difluorophenoxy)-1H-pyrrolo[2,3-b]pyridin-3-yl)pyrimidin-2-amine hydrochloride;
I-23: 4-(4-(3-(trifluoromethyl)phenoxy)-1H-pyrrolo[2,3-b]pyridin-3-yl)pyrimidin-2-amine hydrochloride;
I-24: 4-(4-(4-fluorophenoxy)-1H-pyrrolo[2,3-b]pyridin-3-yl)pyrimidin-2-amine hydrochloride;
I-25: 4-(4-phenoxy-1H-pyrrolo[2,3-b]pyridin-3-yl)pyrimidin-2-ol hydrochloride;
I-26: 4-(4-(3-chlorophenoxy)-1H-pyrrolo[2,3-b]pyridin-3-yl)pyrimidin-2-ol hydrochloride;
I-27: 4-(4-(3-fluorophenoxy)-1H-pyrrolo[2,3-b]pyridin-3-yl)pyrimidin-2-ol hydrochloride;
I-28: 4-(4-(3,5-difluorophenoxy)-1H-pyrrolo[2,3-b]pyridin-3-yl)pyrimidin-2-ol hydrochloride;
I-29: 4-(4-(3-(trifluoromethyl)phenoxy)-1H-pyrrolo[2,3-b]pyridin-3-yl)pyrimidin-2-ol hydrochloride;
I-30: 4-(4-(4-fluorophenoxy)-1H-pyrrolo[2,3-b]pyridin-3-yl)pyrimidin-2-ol hydrochloride;
I-31: 6-(4-phenoxy-1H-pyrrolo[2,3-b]pyridin-3-yl)pyrimidin-4-amine hydrochloride;
I-32: 6-(4-(3-chlorophenoxy)-1H-pyrrolo[2,3-b]pyridin-3-yl)pyrimidin-4-amine hydrochloride;
I-33: 6-(4-(3-fluorophenoxy)-1H-pyrrolo[2,3-b]pyridin-3-yl)pyrimidin-4-amine hydrochloride;
I-34: 6-(4-(3,5-difluorophenoxy)-1H-pyrrolo[2,3-b]pyridin-3-yl)pyrimidin-4-amine hydrochloride;
I-35: 6-(4-(3-(trifluoromethyl)phenoxy)-1H-pyrrolo[2,3-b]pyridin-3-yl)pyrimidin-4-amine hydrochloride;
I-36: 6-(4-(4-fluorophenoxy)-1H-pyrrolo[2,3-b]pyridin-3-yl)pyrimidin-4-amine hydrochloride;
I-37: 4-(4-(3-fluorophenoxy)-1H-pyrrolo[2,3-b]pyridin-3-yl)pyrimidin-2-amine;
I-38: 4-(4-(3-fluoro-5-(morpholinomethyl)phenoxy)-1H-pyrrolo[2,3-b]pyridin-3-yl)pyrimidin-2-amine;
I-39: 4-(4-(3-((dimethylamino)methyl)-5-fluorophenoxy)-1H-pyrrolo[2,3-b]pyridin-3-yl)pyrimidin-2-amine;
I-40: 2-(dimethylamino)-N-(4-(4-(3-fluorophenoxy)-1H-pyrrolo[2,3-b]pyridin-3-yl)pyrimidin-2-yl)acetamide;
I-41: 4-(4-((5-fluoropyridin-3-yl)oxy)-1H-pyrrolo[2,3-b]pyridin-3-yl)pyrimidin-2-amine;
I-42: 4-(4-(piperidin-4-yloxy)-1H-pyrrolo[2,3-b]pyridin-3-yl)pyrimidin-2-amine; or
I-43: 4-(4-((4-fluorobicyclo[1.1.1]pentan-2-yl)oxy)-1H-pyrrolo[2,3-b]pyridin-3-yl)pyrimidin-2-amine.

### IV. Synthesis

Also disclosed are embodiments of a method for making the disclosed compounds. In some embodiments, the method has a first step according to Scheme 1.

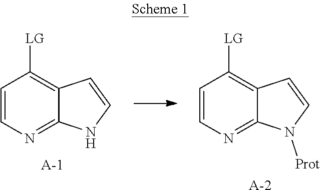

With respect to Scheme 1, LG is a leaving group, suitable for facilitating addition of the phenoxy moiety. Exemplary leaving groups include, but are not limited to, halogen, such as Cl, Br, F or I, typically, Cl. And Prot is a protecting group suitable to protect the nitrogen during the reaction to add the phenoxy group. Exemplary Prot groups include, but are not limited to, silica protecting groups, such as trimethylsilylethoxymethyl (SEM). A person of ordinary skill in the art understands which protecting groups can be used in different circumstances, and also knows suitable methods for introducing and removing such protecting groups. Additional information can be found in Greene's Protective Groups in Organic Synthesis, 5^{th} Edition, by Peter Wuts, published by Wiley.

Also with respect to Scheme 1, compound A-1 is treated with a suitable base, such as a hydride base, typically, NaH, and a Prot-X compound where X is a suitable leaving group, such as a halogen, typically, Cl or Br. The mixture is allowed to react for a time period and at a temperature suitable to facilitate the reaction proceeding to completion. After a suitable work up, compound A-2 is isolated by a suitable technique, such as column chromatography.

The synthesis may proceed with a second step according to Scheme 2.

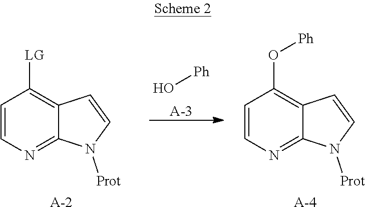

Compound A-2 is treated with hydroxy compound A-3 to form compound A-4. A person of ordinary skill in the art understands that, although compound A-3 is shown as a phenol compound, compound A-3 could instead be a heteroaryl-OH compound, or an aliphatic-OH compound, and in any embodiments, compound A-3 may be unsubstituted or substituted. The reaction may proceed in the presence of a suitable catalyst, such as a palladium catalyst. Exemplary catalysts include, but are not limited to, Pd₂(dba)₃. The reaction may also proceed in the presence of a phosphine ligand, such as dicyclohexyl[2′,4′,6′-tris(propan-2-yl)[1,1′-biphenyl]-2-yl]phosphane (XPhos), and/or a base, such as a carbonate base, typically K₂CO₃. The reaction may be performed in an aprotic solvent, such as toluene or xylene. And the mixture may be allowed to react for a time period and at a temperature suitable to facilitate the reaction proceeding to completion, such as 60° C. to 150° C., and may be performed at about 110° C.

A third step of the synthesis may proceed according to Scheme 3.

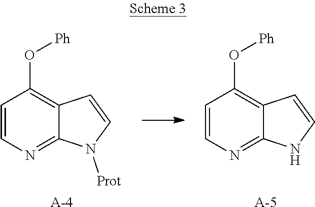

Compound A-4 is deprotected to form compound A-5. When Prot is SEM, the deprotection reaction occurs in the presence of trifluoroacetic acid (TFA) followed by treatment with a suitable base, such as a carbonate base, for example, potassium or sodium carbonate, or potassium or sodium hydrogen carbonate, or a combination thereof. A person of ordinary skill in the art understands that different Prot moieties require different conditions for deprotection, and such deprotection strategies are within the knowledge of a person of ordinary skill in the art. The mixture may be allowed to react for a time period and at a temperature suitable to facilitate the reaction proceeding to completion, such as at ambient temperature. Typically, the product is isolated by a suitable technique, such as column chromatography.

The synthesis may proceed by a fourth step according to Scheme 4.

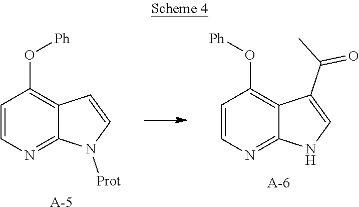

Compound A-5 is treated with a suitable acylating agent to form compound A-6. The acylating agent may be any agent suitable to introduce the acyl moiety onto the ring. Exemplary acylating agents include, but are not limited to, acetic anhydride, or an acyl halide, for example, acyl chloride or acyl bromide. The reaction may proceed in the presence of an additional reagent, such as trifluoroacetic acid, and/or a Lewis acid such as AlCl₃ or FeCl₃. The mixture may be allowed to react for a time period and at a temperature suitable to facilitate the reaction proceeding to completion, such as at about 75° C. to 120° C. or about 90° C. for from 12 to 24 hours. The product may be isolated by a suitable technique, such as column chromatography.

The reaction may further proceed via a fifth step according to Scheme 5.

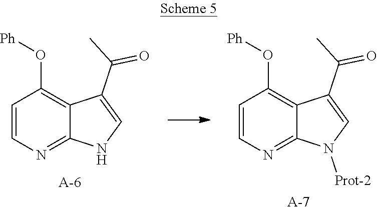

Compound A-6 is treated with a protecting agent to form Compound A-7. The Prot-2 moiety can be any protecting group suitable to protect the NH through the next steps of the synthesis. In some embodiments, Prot-2 is an optionally substituted benzenesulfonyl moiety and therefore compound A-6 is treated with a Prot-2-X compound, where X is a suitable leaving group, such as a halide, for example chloride.

In any embodiments, the reaction may proceed in the presence of a suitable base, such as an organic base, for example, triethylamine or diisopropylethylamine (DIPEA), or pyridine, an inorganic base, such as a hydride base (for example, NaH), a carbonate base (for example, lithium, potassium, sodium or calcium carbonate or hydrogen carbonate), a hydroxide base (for example sodium, lithium or potassium hydroxide), or a combination thereof. The mixture may be allowed to react for a time period and at a temperature suitable to facilitate the reaction proceeding to completion. The product may be isolated by a suitable technique, such as column chromatography.

The synthetic route may further comprise a step according to Scheme 6,

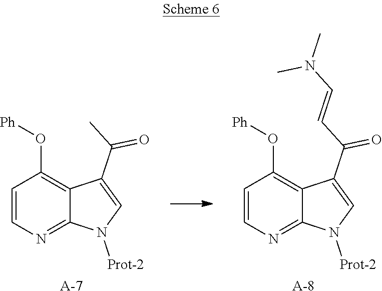

Compound A-7 is treated with N,N-dimethylformamide dimethyl acetal (DMF-DMA) for form compound A-8. The mixture may be allowed to react for a time period and at a temperature suitable to facilitate the reaction proceeding to completion, such as heating at 75° C. to 110° C., or about 90° C. for 6 to 18 hours. After cooling the product may be isolated by a suitable technique, such as column chromatography.

The synthesis may further include a step according to Scheme 7 to product a disclosed compound.

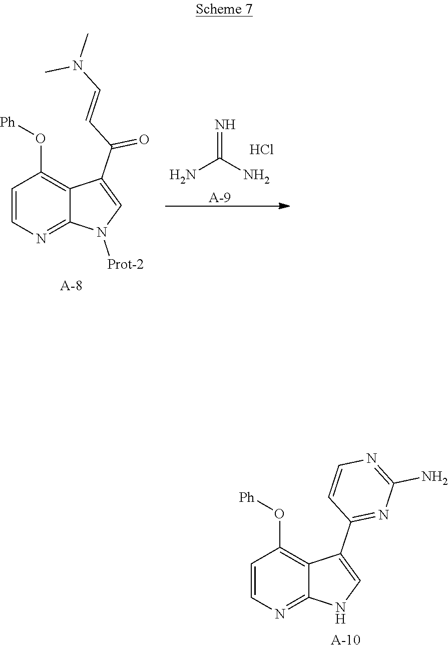

With respect to Scheme 7, compound A-8 is treated with guanidine compound A-9 to form compound A-10. The reaction may proceed in the present of a suitable base, such as an organic base, for example, triethylamine or diisopropylethylamine (DIPEA), or pyridine, an inorganic base, a carbonate base (for example, lithium, potassium, sodium or calcium carbonate or hydrogen carbonate), a hydroxide base (for example sodium, lithium or potassium hydroxide), or a combination thereof. The mixture may be allowed to react for a time period and at a temperature suitable to facilitate the reaction proceeding to completion, such as heating at 90° C. to 130° C., or about 110° C. for 12 to 24 hours. After cooling the product may be isolated by a suitable technique, such as column chromatography.

Additional and/or alternative methods are provided in the Examples.

### V. Pharmaceutical Compositions and Methods of Treatment

Disclosed herein are methods of treating a subject with one or more compounds provided herein. In some embodiments, the subject has cancer. In some examples, the subject has breast cancer, for example, triple negative breast cancer, such as basal-like breast cancer. In other examples, the subject has ovarian cancer, pancreatic cancer, or hepatocellular carcinoma. In further examples, the subject has a cancer that expresses or overexpresses CDK2.

In some embodiments, the disclosed compounds are specific inhibitors of CDK2, for example compared to another CDK, such as one or more of CDK1, CDK4, CDK5, CDK6, CDK7, and CDK9; however, this is not necessarily required for a disclosed compound to be effective for the methods described herein. In some examples, a disclosed compound has a decreased IC₅₀ for inhibition of CDK2 compared to one or more of CDK1, CDK4, CDK5, CDK6, CDK7, and CDK9, for example, an IC₅₀ for CDK2 that is decreased by at least 10%, 25%, 50%, 75%, 2-fold, 5-fold, 10-fold, 20-fold, 50-fold, 100-fold, or more, compared to the IC₅₀ for one or more of CDK1, CDK4, CDK5, CDK6, CDK7, and CDK9. In other examples, a disclosed compound has a decreased IC₅₀ for CDK2 compared to a control CDK inhibitor, for example, an IC₅₀ for CDK2 that is decreased by at least 10%, 25%, 50%, 75%, 2-fold, 5-fold, 10-fold, 20-fold, 50-fold, 100-fold, or more, compared to the control CDK inhibitor. Exemplary methods of determining the IC50 of a compound for a CDK are described in Example 6. Other methods of assessing CDK inhibition include target knockout (e.g., utilizing siRNA or CRIPSR) mediated cell viability assays in presence or absence of an inhibitor.

This disclosure includes pharmaceutical compositions including at least one of the compounds described herein for use in human or veterinary medicine. Embodiments of pharmaceutical compositions include a pharmaceutically acceptable carrier and/or excipient and at least one of the disclosed compounds. Useful pharmaceutically acceptable carriers and excipients are known in the art.

The pharmaceutical compositions including one or more of the compounds disclosed herein may be formulated in a variety of ways depending, for example, on the mode of administration and/or the subject or disorder to be treated. For example, pharmaceutical compositions may be formulated as pharmaceutically acceptable salts. As another example, parenteral formulations may comprise injectable fluids that are pharmaceutically and physiologically acceptable fluid vehicles such as water, physiological saline, other balanced salt solutions, aqueous dextrose, glycerol or the like. Excipients may include, for example, nonionic solubilizers, such as Cremophor®, or proteins, such as human serum albumin or plasma preparations. In some examples, the pharmaceutical composition to be administered may also contain non-toxic auxiliary substances, such as wetting or emulsifying agents, preservatives, and pH buffering agents and the like, for example, sodium acetate or sorbitan monolaurate.

Routes of administration include but are not limited to oral and parenteral routes, such as intravenous, intraperitoneal, rectal, topical, ophthalmic, intranasal, and transdermal. The compound may also be delivered intramuscularly or subcutaneously. In particular examples, the compound is administered orally. In other specific examples, the compound is administered intravenously. To extend the time during which the compound is available to inhibit or treat a condition, the compound can be provided as an implant, an oily injection, a liposome, or as a particulate system. The particulate system can be a microparticle, a microcapsule, a microsphere, a nanoparticle, a nanocapsule, or similar particle.

The dosage form of the pharmaceutical composition can be determined, at least in part, by the mode of administration chosen. For example, in addition to injectable fluids, topical or oral formulations may be employed. Topical preparations may include eye drops, ointments, sprays, and the like. Oral formulations may be liquid (e.g., syrups, solutions or suspensions), or solid (e.g., powders, pills, tablets, or capsules). For solid compositions, non-toxic solid carriers include but are not limited to pharmaceutical grade mannitol, lactose, starch, or magnesium stearate.

Pharmaceutical compositions for oral use can also be formulated, for example, as tablets, troches, lozenges, aqueous or oily suspensions, dispersible powders or granules, emulsion hard or soft capsules, or syrups or elixirs. Such compositions may contain one or more agents selected from the group of sweetening agents, flavoring agents, coloring agents and preserving agents. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatin or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monostearate or glyceryl distearate may be employed. Pharmaceutical compositions for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium such as peanut oil, liquid paraffin or olive oil.

In some embodiments, a carrier for preparing an oral formulation of a disclosed compound includes Tween 80, glycerol, and a cyclodextrin (such as sulfobutylether-β-cyclodextrin (SBE-β-CD; Captisol®). In one example, the carrier includes 0.1% (v/v) Tween 80, and 99.9% (v/v) of 0.5% (w/v) of methylcellulose in water. In other embodiments, a carrier for preparing an oral formulation of a disclosed compound includes a non-ionic surfactant (e.g., caprylocaproyl polyoxyl-8 glycerides (Labrasol®), an oil (e.g., transesterified ethoxylated vegetable oil (e.g., Labrafil®), and a solubilizer (such as diethylene glycol monoethyl ether (e.g., Transcutol®). In a specific example, the carrier includes 40% (v/v) Labrasol, 40% (v/v) Labrafil, and 20% (v/v) Transcutol. Other carriers that can be used in formulations of the disclosed compounds include polyethylene glycol (e.g., PEG 400), propylene glycol; water (e.g., sterile water), and N,N-dimethylacetamide (DMA). For IV formulation, an exemplary carrier includes 5% (v/v) DMS), 2.5% (v/v) absolute ethanol, 2.5% (v/v) Solutol, and 90% saline.

The disclosed compounds can be conveniently presented in unit dosage form and prepared using techniques known to one of skill in the art. Such techniques include the step of bringing into association the active ingredient and the pharmaceutical carrier(s) or excipient(s). The formulations may be included in unit-dose or multi-dose containers, for example, sealed ampoules and vials, and may be stored in a dried condition requiring only the addition of a sterile liquid carrier, for example, water or saline for injections, immediately prior to use. In certain embodiments, unit dosage formulations are those containing a dose or unit, or an appropriate fraction thereof, of the administered ingredient.

The amount of the compound that will be effective depends on the nature of the disorder or condition to be treated, as well as the stage of the disorder or condition. Effective amounts can be determined by in vitro studies, animal studies, and clinical techniques. The precise dose of the compounds to be included in the formulation will also depend on the route of administration, and should be decided according to the judgment of the health care practitioner and each subject's circumstances. An example of such a dosage range is 1 μg/kg to 200 mg/kg body weight (for example, about 5 μg/kg to 1 mg/kg, about 10 μg/kg to 5 mg/kg, about 100 μg/kg to 20 mg/kg, about 0.2 to 100 mg/kg, about 0.5 to 50 mg/kg, about 1 to 25 mg/kg, about 5 to 75 mg/kg, about 50 to 150 mg/kg, or about 100 to 200 mg/kg) in single or divided doses. For example, a suitable dose may be about 0.1 mg/kg, about 0.2 mg/kg, about 0.5 mg/kg, about 0.75 mg/kg, about 1 mg/kg, about 2 mg/kg, about 5 mg/kg, about 7.5 mg/kg, about 10 mg/kg, about 15 mg/kg, about 20 mg/kg, about 25 mg/kg, about 30 mg/kg, about 40 mg/kg, about 50 mg/kg, about 75 mg/kg, about 100 mg/kg, about 150 mg/kg, or about 200 mg/kg.

One or more doses of the compound can be administered to a subject. For example, the compound can be administered three times per day, twice per day, daily, every other day, twice per week, weekly, every other week, every three weeks, monthly, or less frequently. In some examples, the compound may be administered in cycles, for example, at a set interval (such as weekly or daily) for a set number of intervals, followed by a rest period, then repeated one or more times.

The specific dose level and frequency of dosage for any particular subject may be varied and will depend upon a variety of factors, including the disorder being treated, the specific compound being administered, the age, body weight, general health, sex and diet of the subject, mode and time of administration, and so on.

In some embodiments, the subject being treated has a solid tumor. Examples of solid tumors include sarcomas (such as fibrosarcoma, myxosarcoma, liposarcoma, chondrosarcoma, osteosarcoma, soft tissue sarcoma, and other sarcomas), synovioma, mesothelioma, Ewing sarcoma, leiomyosarcoma, rhabdomyosarcoma, colon cancer, colorectal cancer, peritoneal cancer, esophageal cancer (such as esophageal squamous cell carcinoma), pancreatic cancer, breast cancer (including basal breast carcinoma, ductal carcinoma and lobular breast carcinoma), endometrial cancer, lung cancer (such as non-small cell lung cancer), ovarian cancer, prostate cancer, liver cancer (including hepatocellular carcinoma), gastric cancer, squamous cell carcinoma (including head and neck squamous cell carcinoma), basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, hepatoma, bile duct carcinoma, choriocarcinoma, Wilms tumor, cervical cancer, fallopian tube cancer, testicular tumor, seminoma, bladder cancer (such as renal cell cancer), melanoma, and CNS tumors (such as a glioblastoma, astrocytoma, medulloblastoma, diffuse intrinsic pontine glioma, craniopharyrgioma, ependymoma, pinealoma, hemangioblastoma, acoustic neuroma, oligodendroglioma, meningioma, neuroblastoma and retinoblastoma). Solid tumors also include tumor metastases (for example, metastases to the lung, liver, brain, or bone).

In other examples, the subject has a hematological malignancy. Examples of hematological malignancies include leukemias, including acute leukemias (such as 11q23-positive acute leukemia, acute lymphocytic leukemia (ALL), T-cell ALL, acute myelocytic leukemia, acute myelogenous leukemia (AML), and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), lymphoblastic leukemia, polycythemia vera, lymphoma, diffuse large B cell lymphoma, Burkitt lymphoma, T cell lymphoma, follicular lymphoma, mantle cell lymphoma, Hodgkin disease, non-Hodgkin lymphoma, multiple myeloma, Waldenstrom macroglobulinemia, heavy chain disease, myelodysplastic syndrome, hairy cell leukemia, and myelodysplasia.

In particular examples, the subject has breast cancer, such as triple negative breast cancer. In other examples, the subject has hepatocellular carcinoma, ovarian cancer, ER-positive breast cancer, or pancreatic cancer.

In some examples, the subject with cancer is also treated with surgery, radiation therapy, chemotherapeutic agents, immunotherapy, or any combination thereof. A skilled clinician can select an appropriate combination of additional treatments with the compounds provided herein, based on the type of cancer being treated.

### VI. EXAMPLES

The following examples are provided to illustrate certain features and/or embodiments. These examples should not be construed to limit the disclosure to the particular features or embodiments described.

### Example 1

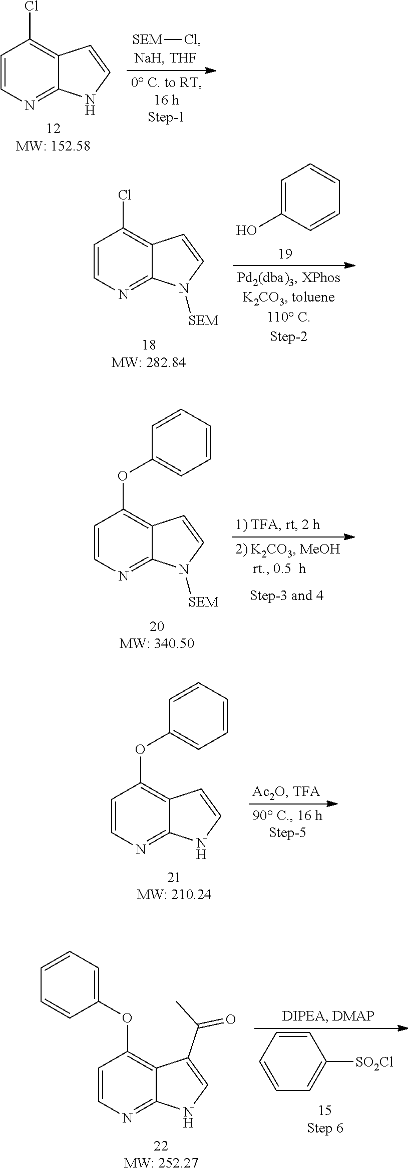

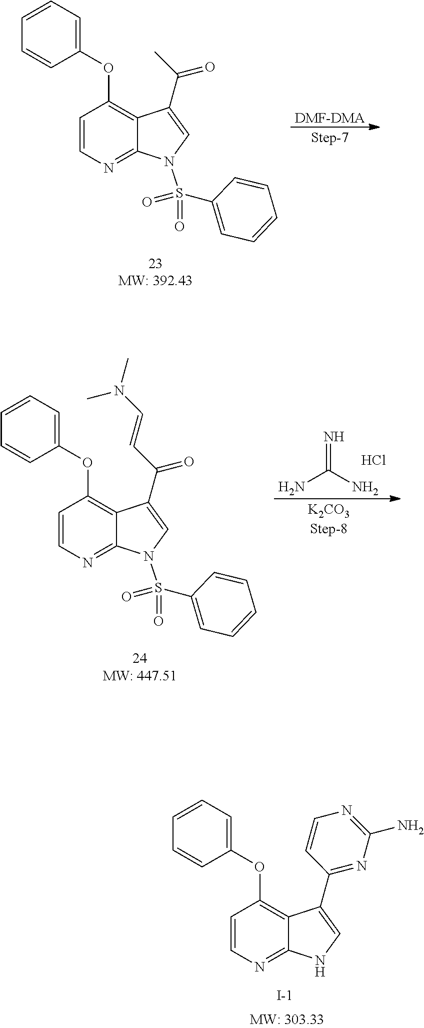

Step-1: To a stirred suspension of NaH (2.04 g, 0.08520 mol) in THF (10 vol.) under nitrogen at 0° C. was added compound 12 (10 g, 0.06553 mol), and the reaction mixture was stirred at same temperature for additional one hour. Then the SEM-Chloride (17.3 mL, 0.0982) was added in drops to the mixture and the mixture was stirred at room temperature overnight. The resulting mixture was cooled to 0° C., quenched with water (100 ml), and extracted with dichloromethane (DCM) (2×100 ml). The organic layer was washed with water (50 mL), brine (50 mL), dried over anhydrous Na₂SO₄, and the solvent was evaporated under reduced pressure. The crude product was purified by column chromatography to obtain compound 18 (14 g) as a pale yellow liquid.

Step-2: To a stirred solution of compound 18 (5 g, 0.01767 mol) in toluene (10 vol.) under nitrogen were added compound 19 (2.16 g, 0.02298 mol) and K₂CO₃ (5.375 g, 0.03889 mol) at room temperature, and the reaction mixture was degassed with nitrogen for 10 minutes. Pd₂(dba)₃ (0.809 g, 0.00088 mol) and XPhos (0.842 g, 0.00176 mol) were added, and the resulting mixture was heated at 110° C. for 16 hours. The reaction was cooled to room temperature, filtered through a Celite bed, and the filtrate was concentrated under reduced pressure. The resulting residue was diluted with water and extracted with DCM (3×100 mL). The organic layer was washed with water (100 mL), brine (100 mL), dried over anhydrous Na₂SO₄ and the solvent was evaporated under reduced pressure. The crude 20 (6 g) was used for next step without further purification.

Step-3: To a stirred solution of compound 20 (5 g, 0.01468 mol) in DCM (10 vol.) was added trifluoroacetic acid (TFA) (5 vol.) at room temperature, and the reaction mixture was stirred at the same temperature for 2 hours. The reaction mixture was quenched with NaHCO₃solution (100 mL) and extracted with DCM (3×200 mL). The organic layer was washed with water (100 mL), brine (150 mL), dried over anhydrous Na₂SO₄ and the solvent was evaporated under reduced pressure. The crude product was dissolved in methanol (10 vol.) and added K₂CO₃ (6 g, 0.04405 mol) was added at room temperature for 30 minutes. The solid was filtered off and the filtrate was concentrated. The crude product was purified by column chromatography to obtain compound 21 (1.8 g) as a brown solid.

Step-4: To a stirred solution of compound 21 (1.8 g, 0.00856 mol) in TFA (20 vol.) under nitrogen was added acetic anhydride (4.85 ml, 0.05136 mol) at room temperature. The resulting mixture was heated at 90° C. for 12 hours. The reaction mixture was cooled to room temperature and the solvent was evaporated under reduced pressure. The resulting mixture was quenched with Na₂CO₃ solution (80 mL) until the effervescence has ceased. The aqueous layer was extracted with DCM (2×100 ml). The organic layer was washed with water (50 mL), brine (50 mL), dried over anhydrous Na₂SO₄, and the solvent was evaporated under reduced pressure. The crude residue was purified by column chromatography to obtain compound 22 (1.5 g) as a brown solid.

Step-5: To a stirred solution of compound 22 (1.5 g, 0.00594 mol) in DCM (10 vol.) under nitrogen were added DIPEA (5.17 ml, 0.02973 mol), benzenesulfonyl chloride (compound 15) (1.26 g, 0.007135 mol) and DMAP (0.072 g, 0.00059 mol) at room temperature. The mixture was stirred at room temperature for 16 hours and the solvent then was evaporated under vacuum. The resultant residue was quenched with water (20 ml) and extracted with DCM (2×100 ml). The organic layer washed with water (50 mL), brine (50 mL), dried over anhydrous Na₂SO₄, and the solvent was evaporated under reduced pressure. The crude residue was purified by column chromatography to obtain compound 23 (1.4 g) as off white solid.

Step-6: To a stirred solution of compound 23 (1.4 g, 0.00356 mol) in DMF (8 vol.) was added DMF-DMA (2.84 ml, 0.02140 mol) at room temperature and the mixture was heated at 90° C. for 12 hours. The reaction mixture was cooled to room temperature and the solvent was evaporated under vacuum. the crude product 24 (1.7 g) was taken for the next step without further purification.

Step-7: To a stirred solution of compound 24 (1.7 g, 0.003798 mol) in 2-methoxyethanol (5 vol.) under nitrogen were added K₂CO₃ (1.15 g, 0.00835 mol) and guanidine hydrochloride (0.544 g, 0.005698 mol) at room temperature. The mixture was heated to 110° C. for 16 hours. The reaction mixture was cooled to room temperature and the solvent was evaporated under reduced pressure. The resulting mixture was diluted with water (20 ml) and extracted with DCM (2×100 ml). The organic layer washed with water (100 mL), brine (100 mL) and dried over anhydrous Na₂SO₄, and the solvent was evaporated under reduced pressure. The crude product was purified by column chromatography to get compound I-1 (60 mg) as yellow solid.

### Analytics:

¹H-NMR (400 MHz, DMSO-d6): δ 12.33 (s, 1H), 8.13 (dd, J=8.28, 5.44 Hz, 2H), 8.03 (d, J=2.72 Hz, 1H), 7.48 (t, J=8.20 Hz, 2H), 7.29-7.21 (m, 4H), 6.42 (d, J=5.40 Hz, 1H), 6.34 (s, 2H), LCMS: (A: 0.1% HCOOH in H₂O, B: ACN; Flow Rate: 1.5 mL/min, Column: Atlantis DC18 (50×4.6 mm, 5 μm), +ve mode), RT=2.169 min, 95.18%, 304.1 (M+1).

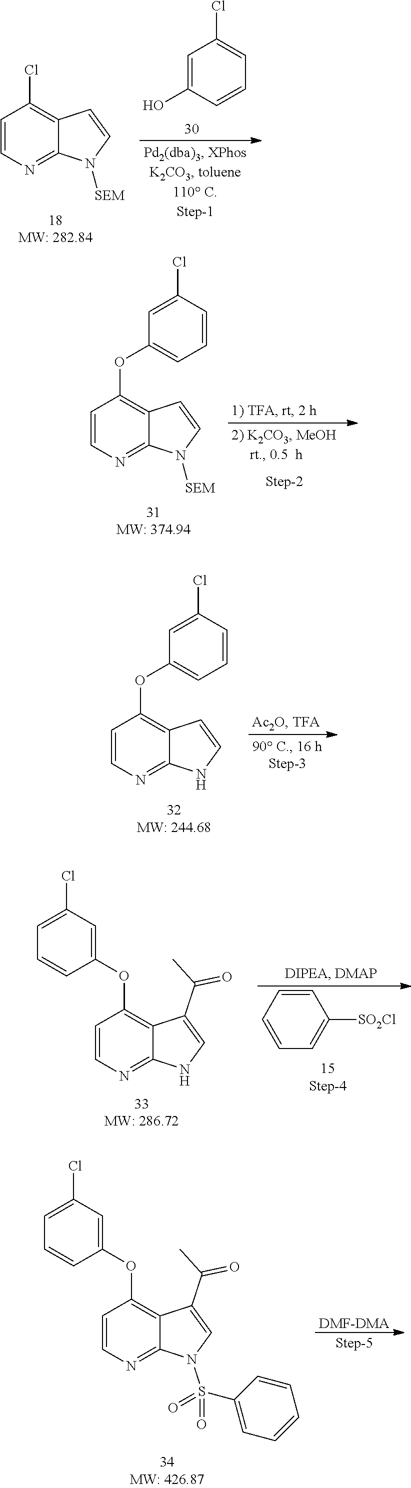

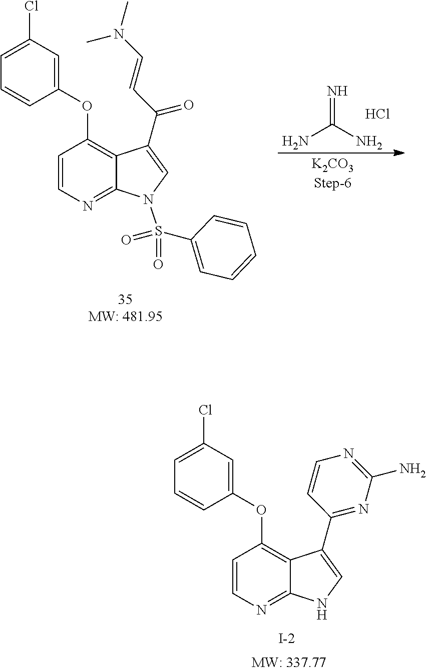

Step-1: To a stirred solution of compound 18 (5 g, 0.01767 mol) in toluene (10 vol.) were added compound 30 (2.9 g, 0.02298 mol) and K₂CO₃ (5.375 g, 0.03889 mol) at room temperature and the reaction mixture was degassed with nitrogen for 10 minutes. Pd₂(dba)₃ (0.809 g, 0.00088 mol) and XPhos (0.842 g, 0.00176 mol) were added and the mixture was heated at 110° C. for 16 hours. The reaction mixture was cooled to room temperature and filtered through a pad of Celite. The filtrate was evaporated under vacuum. The resulting residue was diluted with water and extracted with DCM (3×200 mL). The combined organic layer was washed with water (100 mL), brine (100 mL) and dried with Na₂SO₄ and the solvent was evaporated under reduced pressure. The crude was purified by column chromatography to obtain compound 31 (3 g) as pale brown liquid.

Step-2: To a stirred solution of compound 31 (3 g, 0.008001 mol) in DCM (10 vol.) was added TFA (5 vol.) at 80° C. and the reaction mixture was stirred at 80° C. for 16 hours. Then the reaction was quenched with a saturated solution of NaHCO₃ (20 mL) and extracted with DCM (3×150 mL). The combined organic layer was washed with water (50 mL), brine (50 mL) and dried with Na₂SO₄ and the solvent was evaporated under reduced pressure. The crude compound was dissolved in methanol (10 vol) and K₂CO₃ (3.3 g, 0.0240 mol) was added the solution at room temperature. After being stirred at room temperature for 30 minutes, the solid was filtered off and the filtrate was concentrated. The crude compound was purified by column chromatography to obtain compound 32 (600 mg) as a brown solid.

Step-3: To a stirred solution of compound 32 (0.5 g, 0.00204 mol) in TFA (20 vol) under nitrogen was added acetic anhydride (1.16 ml, 0.0122 mol) at room temperature. The mixture was heated to 90° C. for 12 hours. The reaction mixture was cooled to room temperature and the solvent was evaporated under vacuum. The resultant residue was quenched with Na₂CO₃ (50 mL) solution until the effervescence has ceased. The aqueous layer was extracted with DCM (2×100 ml). The combined organic layer was washed with water (50 mL), brine (50 mL), dried over anhydrous Na₂SO₄, and the solvent was evaporated under reduced pressure. The crude compound was purified by column chromatography to get compound 33 (0.260 g) as a brown solid.

Step-4: To a stirred solution of compound 33 (0.252 g, 0.000880 mol) in DCM (10 vol.) were added DIPEA (0.8 ml, 0.00440 mol), benzenesulfonyl chloride (0.187 g, 0.00105 mol) and DMAP (0.011 g, 0.000088 mol) at room temperature. The mixture was stirred at the same temperature for 16 hours, and then the solvent was evaporated under vacuum. The resultant residue was quenched with water (50 ml) and extracted with DCM (2×100 ml). The combined organic layer was washed with water (50 mL), brine (50 mL) and dried over anhydrous Na₂SO₄, and the solvent was evaporated under reduced pressure. The crude residue was purified by column chromatography to obtain compound 34 (0.250 g) as an off white solid.

Step-5: To a stirred solution of compound 34 (0.250 g, 0.00356 mol) in DMF (8 vol.) was added DMF-DMA (4 vol.) at room temperature and the mixture was heated at 90° C. for 12 hours. The reaction mixture was cooled to room temperature and the solvent was evaporated under vacuum. The crude product 35 (0.260 g) was used for next step without further purification.

Step-6: To a stirred solution of compound 35 (0.250 g, 0.000518 mol) in 2-methoxyethanol (5 vol.) under nitrogen was added K₂CO₃ (0.180 g, 0.00129 mol) followed by guanidine hydrochloride (0.080 g, 0.000829 mol) at room temperature. The resulting mixture was heated at 110° C. for 16 hours. The reaction mixture was cooled to room temperature and the solvent was evaporated under vacuum. The resultant residue was diluted with water (100 ml) and extracted with DCM (2×100 ml). The combined organic layer was washed with water (50 mL), brine (50 mL), dried over anhydrous Na₂SO₄, and the solvent was evaporated under reduced pressure. The crude compound was purified by column chromatography to obtain compound I-2 (25 mg) as off-white solid.

### Analytics:

¹H-NMR (400 MHz, DMSO-d6): 12.39 (s, 1H), 8.20 (d, J=5.20 Hz, 111), 8.12 (d, J=5.60 Hz, 1H), 8.03 (d, J=2.00 Hz, 111), 7.45 (t, J=8.00 Hz, 111), 7.31 (t, J=8.00 Hz, 111), 7.14 (d, J=5.60 Hz, 2H), 6.56 (d, J=5.60 Hz, 111), 6.33 (s, 211);

LCMS: (A: 0.1% HCOOH in H₂O, B: ACN; Flow Rate: 1.5 mL/min, Column: Atlantis DC18 (50 15×4.6 mm, 5 m), +ve mode), RT=1.960 min, 97.25%, 338.1 (M+1).

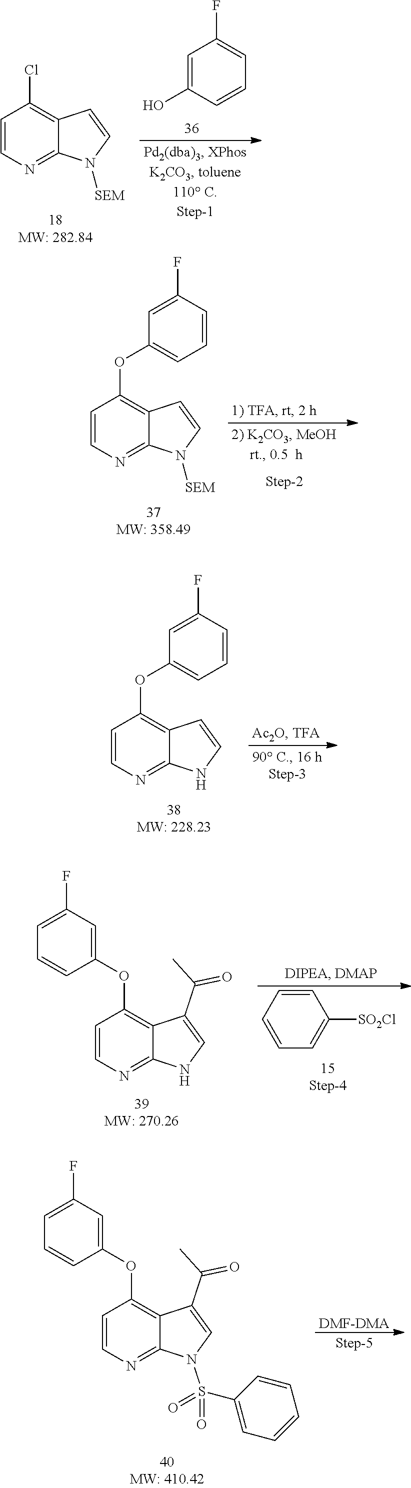

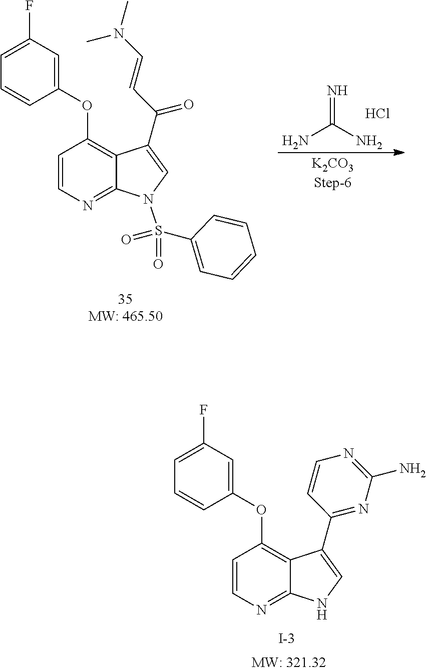

Step-1: To a stirred solution of compound 18 (4 g, 0.01414 mol) in toluene (10 vol.) were added compound 36 (2.01 g, 0.0183 mol) and K₂CO₃ (4.3 g, 0.03110 mol) at room temperature and the reaction mixture was degassed with nitrogen for 10 minutes. Pd₂(dba)₃ (0.646 g, 0.000707 mol) and XPhos (0.674 g, 0.0014 mol) were added, and the mixture was heated at 110° C. for 16 hours. The reaction mixture was cooled to room temperature and filtered through a pad of Celite. The filtrate was evaporated under vacuum. The resulting residue was diluted with water and extracted with DCM (3×200 mL). The combined organic layer was washed with water (100 mL), brine (100 mL) and dried with Na₂SO₄ and the solvent was evaporated under reduced pressure. The crude product was purified by column chromatography to obtain compound 37 (3 g) as a pale brown liquid.

Step-2: To a stirred solution of compound 37 (3 g, 0.00836 mol) in DCM (10 vol.) was added TFA (5 vol.) at 80° C., and the mixture was stirred for 16 hours. Then the reaction was quenched with a saturated solution of NaHCO₃ (20 mL) and extracted with DCM (3×150 mL). The combined organic layer was washed with water (50 mL), brine (50 mL) and dried with Na₂SO₄ and the solvent was evaporated under reduced pressure. The crude compound was dissolved in methanol (10 vol) and K₂CO₃ (3.46 g, 0.0250 mol) was added the solution at room temperature. After being stirred at room temperature for 30 minutes, the solid was filtered off and the filtrate was concentrated. The crude compound was purified by column chromatography to obtain compound 38 (810 mg) as a brown solid.

Step-3: To a stirred solution of compound 38 (0.8 g, 0.003505 mol) in TFA (20 vol) under nitrogen was added acetic anhydride (2.14 ml, 0.021 mol) at room temperature. The mixture was heated at 90° C. for 12 hours. The reaction mixture was cooled to room temperature and the solvent was evaporated under vacuum. The resultant residue was quenched with Na₂CO₃ (50 mL) solution until the effervescence has ceased. The aqueous layer was extracted with DCM (2×100 ml). The combined organic layer was washed with water (50 mL), brine (50 mL), dried over anhydrous Na₂SO₄, and the solvent was evaporated under reduced pressure. The crude compound was purified by column chromatography to obtain compound 39 (0.460 g) as a brown solid.

Step-4: To a stirred solution of compound 39 (0.450 g, 0.00165 mol) in DCM (10 vol.) were added DIPEA (1.4 ml, 0.00832 mol), benzenesulfonyl chloride (compound 15) (0.353 g, 0.00199 mol) and DMAP (0.021 g, 0.000166 mol) at room temperature. The mixture was stirred at the same temperature for 16 hours and the solvent was evaporated under vacuum. The resultant residue was quenched with water (50 ml) and extracted with DCM (2×100 ml). The combined organic layer was washed with water (50 mL), brine (50 mL) and dried over anhydrous Na₂SO₄, and the solvent was evaporated under reduced pressure. The crude product was purified by column chromatography to get the compound 40 (0.4 g) as an off white solid.

Step-5: To a stirred solution of compound 40 (0.400 g, 0.000974 mol) in DMF (8 vol.) was added DMF-DMA (4 vol.) at room temperature and the mixture was heated at 90° C. for 12 hours. The reaction mixture was cooled to room temperature and the solvent was evaporated under vacuum. The crude product 41 (0.410 g) was used for next step without further purification.

Step-7: To a stirred solution of compound 41 (0.400 g, 0.000859 mol) in 2-methoxyethanol (5 vol.) under nitrogen was added K₂CO₃ (0.310 g, 0.00223 mol) followed by guanidine hydrochloride (0.099 g, 0.001034 mol) at room temperature. The resulting mixture was heated at 110° C. for 16 hours. The reaction mixture was cooled to room temperature and the solvent was evaporated under vacuum. The resultant residue was diluted with water (100 ml) and extracted with DCM (2×100 ml). The combined organic layer was washed with water (50 mL), brine (50 mL), dried over anhydrous Na₂SO₄, and the solvent was evaporated under reduced pressure. The crude compound was purified by column chromatography to obtain compound I-3 (80 mg) as an off-white solid.

### Analytics:

¹H-NMR (400 MHz, DMSO-d6): δ 12.38 (s, 1H), 8.20 (d, J=5.20 Hz, 1H), 8.12 (d, J=5.20 Hz, 1H), 8.03 (d, J=2.40 Hz, 111), 7.46 (q, J=8.00 Hz, 1H), 7.14-7.01 (m, 4H), 6.58 (d, J=5.60 Hz, 1H), 6.34 (s, 2H).

LCMS: (A: 0.1% HCOOH in H₂O, B: ACN; Flow Rate: 1.5 mL/min, Column: Atlantis DC18 (50×4.6 mm, 5 m), +ve mode), RT=1.867 min, 97.25%, 322.1 (M+1).

Compound I-6 was prepared by the same method but using 4-fluorophenol in step 1 in place of 3-fluorophenol.

### Example 4

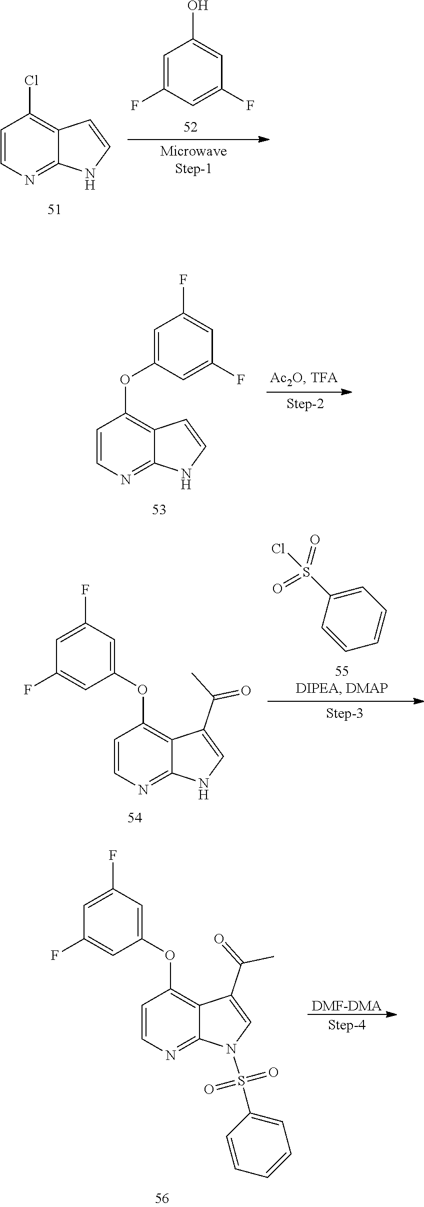

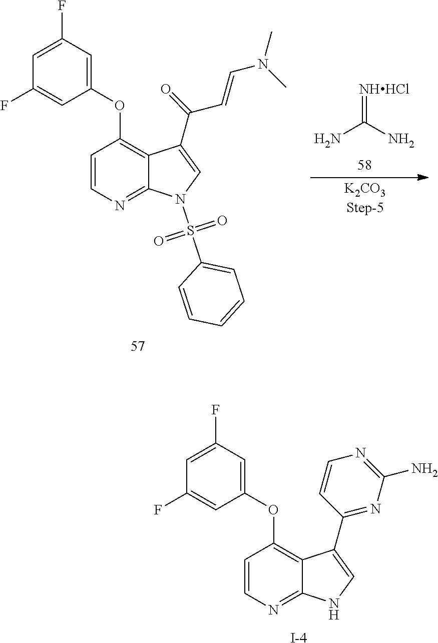

### Step-1: Synthesis of 4-(3,5-difluorophenoxy)-1H-pyrrolo[2,3-b]pyridine

In a sealed tube, compound 51 (10 g), compound 52 (5 eq) treated with K₂CO₃ (1 eq) were added and then the reaction was carried out with microwave at 200° C. for 48 hours. The mixture was cooled to the room temperature. Then crude product was purified by column chromatography on silica (ethyl acetate; Hexane 3:5) to yield compound 53 (4 gm).

### Step-2:-(4-(3, 5-difluorophenoxy)-1H-pyrrolo[2, 3-b] pyridin-3-yl) ethan-1-one

To a stirred solution of compound 53 (3 g, 0.003505 mol) in TFA (20 vol) under nitrogen were added acetic anhydride (2.14 ml, 0.021 mol) at room temperature. The mixture was heated to 90° C. for 12 hours. The reaction mixture was cooled to room temperature and the solvent was evaporated under vacuum. The resultant residue was quenched with Na₂CO₃ (50 mL) solution until the effervescence has ceased. The aqueous layer was extracted with DCM (2×100 ml). The combined organic layer was washed with water (50 mL), brine (50 mL), dried over anhydrous Na₂SO₄, and the solvent was evaporated under reduced pressure. The crude compound was purified by column chromatography to obtain compound 54 (1.2 g) as a brown solid.

### Step-3: Synthesis 1-(4-(3,5-difluorophenoxy)-1-(phenylsulfonyl)-1H-pyrrolo[2,3-b]pyridin-3-yl)ethan-1-one

To a stirred solution of compound 54 (1 g) in DCM (10 vol.) were added DIPEA (5 eq), benzene sulfonyl chloride (1.1 eq) and DMAP (0.1 eq) at room temperature. The mixture was stirred at the same temperature for 12 hours and the solvent was evaporated under vacuum. The resultant residue was quenched with water (50 ml) and extracted with DCM (2×100 ml). The combined organic layer was washed with water (50 mL), brine (50 mL) and dried over anhydrous Na₂SO₄, and the solvent was evaporated under reduced pressure. The crude product was purified by column chromatography to obtain compound 56 (1.2 g) as an off white solid.

### Step-4: Synthesis of (E)-1-(4-(3,5-difluorophenoxy)-1-(phenylsulfonyl)-1H-pyrrolo[2,3-b]pyridin-3-yl)-3-(dimethylamino)prop-2-en-1-one

To a stirred solution of compound 56 (1.2 g) in DMF (8 vol.) was added DMF-DMA (4 vol.) at room temperature and the mixture was heated to 90° C. for 10 hours. The reaction mixture was cooled to room temperature and the solvent was evaporated under vacuum. the crude product compound 57 (0.95 g) was used for next step without further purification.

### Step-5: Synthesis of 4-(4-(3,5-difluorophenoxy)-1H-pyrrolo[2,3-b]pyridin-3-yl) pyrimidin-2-amine

To a stirred solution of compound 57 (0.95 g) in 2-methoxyethanol (5 vol.) under nitrogen were added K₂CO₃ (2.5 eq) followed by guanidine hydrochloride (1.2 eq) at room temperature. The resulting mixture was heated to 110° C. for 16 hours. The reaction mixture was cooled to room temperature and the solvent was evaporated under vacuum. The resultant residue was diluted with water (100 ml) and extracted with DCM (2×100 ml). The combined organic layer was washed with water (50 mL), brine (50 mL), dried over anhydrous Na₂SO₄, and the solvent was evaporated under reduced pressure. The crude compound was purified by column chromatography to obtain compound I-4.

### Example 5

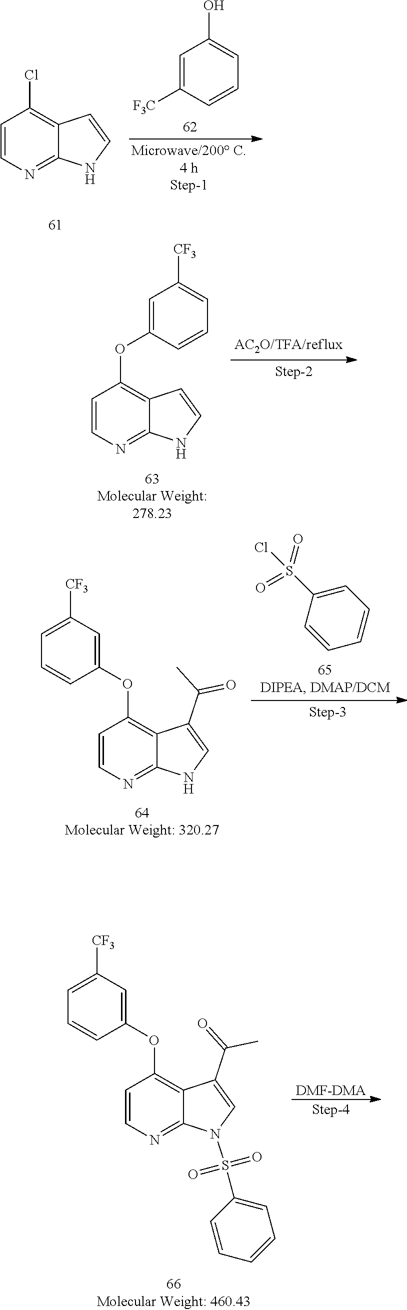

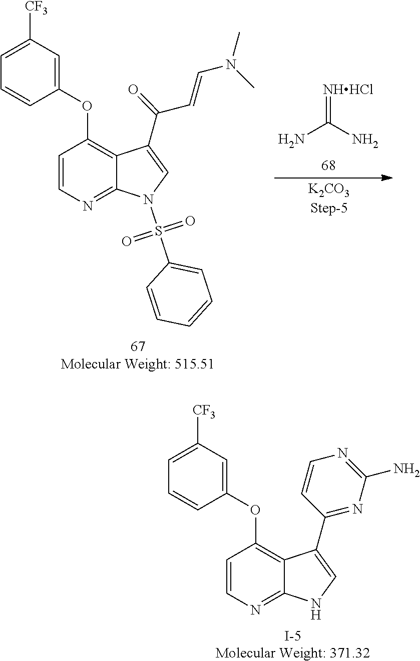

### Step-1

In a sealed tube, compound 61 (15 g), compound 62 (15 vol. Neat reaction) and K₂CO₃ (3 eq) were added and then the reaction was carried out with microwave at 200° C. for 24 hours. The mixture was cooled to the room temperature. Then crude product was purified by column chromatography on silica (ethyl acetate; Hexane 6:4) to yield compound 63 (9.5 gm).

### Step-2

To a stirred solution of compound 63 (9 g, 1 equiv.) in TFA (20 vol) under nitrogen was added acetic anhydride (6 equiv.) at room temperature. The mixture was heated at 90° C. for 24 hours. The reaction mixture was cooled to room temperature and the solvent was evaporated under vacuum. The resultant residue was quenched with aq. Na₂CO₃ (100 mL) solution until the effervescence has ceased. The aqueous layer was extracted with DCM (2×200 ml). The combined organic layer was washed with water (100 mL), brine (100 mL), dried over anhydrous Na₂SO₄, and the solvent was evaporated under reduced pressure. The crude compound was purified by column chromatography to get compound 64 (5.8 g) as a sticky solid.

### Step-3

To a stirred solution of compound 64 (5.5 g) in DCM (10 vol.) were added DIPEA (5 eq), benzene sulfonyl chloride (compound 65) (1.1 eq) and DMAP (0.1 eq, catalytic) at room temperature. The mixture was stirred at the same temperature for 12 hours and the solvent was evaporated under vacuum. The resultant residue was quenched with water (100 ml) and extracted with DCM (2×200 ml). The combined organic layer was washed with water (100 mL), brine (100 mL) and dried over anhydrous Na₂SO₄, and the solvent was evaporated under reduced pressure. The crude was purified by column chromatography to get the compound 66 (6.1 g) as an off white solid.

### Step-4

To a stirred solution of compound 66 (5 g) in DMF (8 vol.) was added DMF-DMA (4 vol.) at room temperature and the mixture was heated to 90° C. for 24 hours. The reaction mixture was cooled to room temperature and the solvent was evaporated under vacuum. The crude product compound 67 (5.8 g) was used for next step without further purification.

### Step-5

To a stirred solution of compound 67 (5.8 g) in 2-methoxyethanol (5 vol.) under nitrogen were added K₂CO₃ (2.5 eq) followed by guanidine hydrochloride (1.2 eq) at room temperature. The resulting mixture was heated to 110° C. for 48 hours. The reaction mixture was cooled to room temperature and the solvent was evaporated under vacuum. The resultant residue was diluted with water (200 ml) and extracted with DCM (2×100 ml). The combined organic layer was washed with water (100 mL), brine (50 mL), dried over anhydrous Na₂SO₄, and the solvent was evaporated under reduced pressure. The crude compound was purified by MS-Based Auto purification (Column: T3 preparative, 5 micron) to obtain compound I-5 (42 mg).

### Example 6

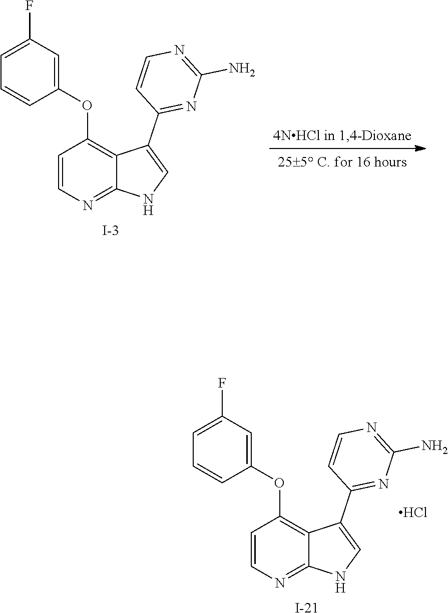

### Synthetic Procedure:

To a solution of 1-3 (50 mg, 0.00014 mol.) in methanol (0.5 mL) at 0° C. was added 4N HCl in 1,4-Dioxane (1 mL) stirred the reaction mass at 25±5° C. for 16 hours. Reaction completion was monitored by TLC analysis. After completion of reaction, the reaction mass was distilled off under reduced pressure, excess HCl was stripped off with 1,4-dioxane and dried under vacuum at 50° C. for about 5 hours to afford I-21 as pale brown solid. Yield: 55 mg.

I-21 (1-3 HCl salt): ¹H NMR (400 MHz, DMSO) δ; 13.16 (s, 1H, —NH), 8.45 (s, 1H), 8.33 (d, J=6.4 Hz, 1H), 8.30 (brs, 2H, —NH₂), 8.27 (d, J=5.6 Hz, 1H), 7.55 (d, J=6.8 Hz, 1H), 7.52-7.46 (m, 1H), 7.25 (dt, J=10.4, 2 Hz, 1H) 7.12 (t, J=8.4 Hz, 1H) 7.07 (dd, J=8.4, 1.6 Hz, 1H), 6.65 (d, J=5.6H, 1H), 5.50 (brs, 3H, HCl).

### Example 7

A series of test compounds were selected for initial screening against CDK isoforms. The results are shown in Table 1. Protein kinase assays were performed with gold standard radioisotope-based assay formats to measuring enzyme activity remaining in presence and absence of inhibitor. In this example, compounds (Table 1) were tested in 10-dose IC₅₀ mode with a 3-fold serial dilution starting at 10 μM. The reactions were carried out at 10 μM ATP, 1-3 was selected for further studies based on potency and selectivity against CDK2 versus other kinases.

TABLE 1
Initial CDK isoform screening

| | IC50 (nM) | | | | | |
|---|---|---|---|---|---|---|
| Target | I-1 | I-2 | I-3 | I-4 | I-5 | I-6 |
| CDK1/cyclin A | 3 | 11 | 6 | | | 4 |
| CDK1/cyclin B | 4 | 16 | 8 | 7 | | |
| CDK2/cyclin A | 3 | 9 | 4 | | | 5 |
| CDK2/cyclin E | 2 | 8 | 4 | 2 | 27 | |
| CDK4/cyclin D1 | 259 | 875 | 414 | 462 | | |
| CDK6/cyclin D1 | 138 | 819 | 285 | | | 196 |
| CDK5/p25 | | | 8 | | | |
| CDK7/cyclin H | | | 18 | | 43 | |
| CDK9/cyclin T1 | | | 7 | | 16 | 3 |

The activity of compound I-3 was compared to that of a panel of CDKs and their respective binding partners (Tables 2 and 3). Pfizer pan-CDK2 inhibitor PF-06873600 and Cyclacel pan-CDK2 inhibitor CYC065 were used as reference compounds.

TABLE 2
Biochemical profiles of compounds
against CDKs and binding partners

| | I-3 | Milciclib | Merioline-3 |
|---|---|---|---|
| Target | IC₅₀ (nM) | IC₅₀ (nM) | IC₅₀ (nM) |
| CDK1/cyclin A | 6 | NT | 5 |
| CDK1/cyclin B | 8 | 398 | 7 |
| CDK2/cyclin A | 4 | 45 | 3 |
| CDK2/cyclin E | 4 | 363 | 2 |
| CDK4/cyclin D1 | 414 | 160 | 182 |
| CDK6/cyclin D1 | 285 | NT | 130 |
| CDK5/p35 | 8 | NT | 3 |
| CDK7/cyclin H | 18 | 150 | NT |
| CDK9/cyclin T1 | 7 | NT | 2 |

NT = not tested

TABLE 3
Biochemical profiles of compounds
against CDKs and binding partners

| | Biochemical IC₅₀ (nM) | | | | |
|---|---|---|---|---|---|
| | CDK1/ | CDK2/ | CDK4/ | CKD9/ | |
| | Cyclin | Cyclin | Cyclin | Cyclin | Major |
| Inhibitor | B | E | D1 | H | CDKs |
| I-3 | 8 | 4 | 414 | 7 | 2, 1, 9 |
| Merioline-3 | 7 | 2 | 182 | 2 | 2, 1, 9 |
| PF-06873600 | 2 | 0.3 | 2 | 43 | 2, 4, 1, 9 |
| CYC065 | 578 | 5 | 21 | 26 | 2, 9 |

### Example 8

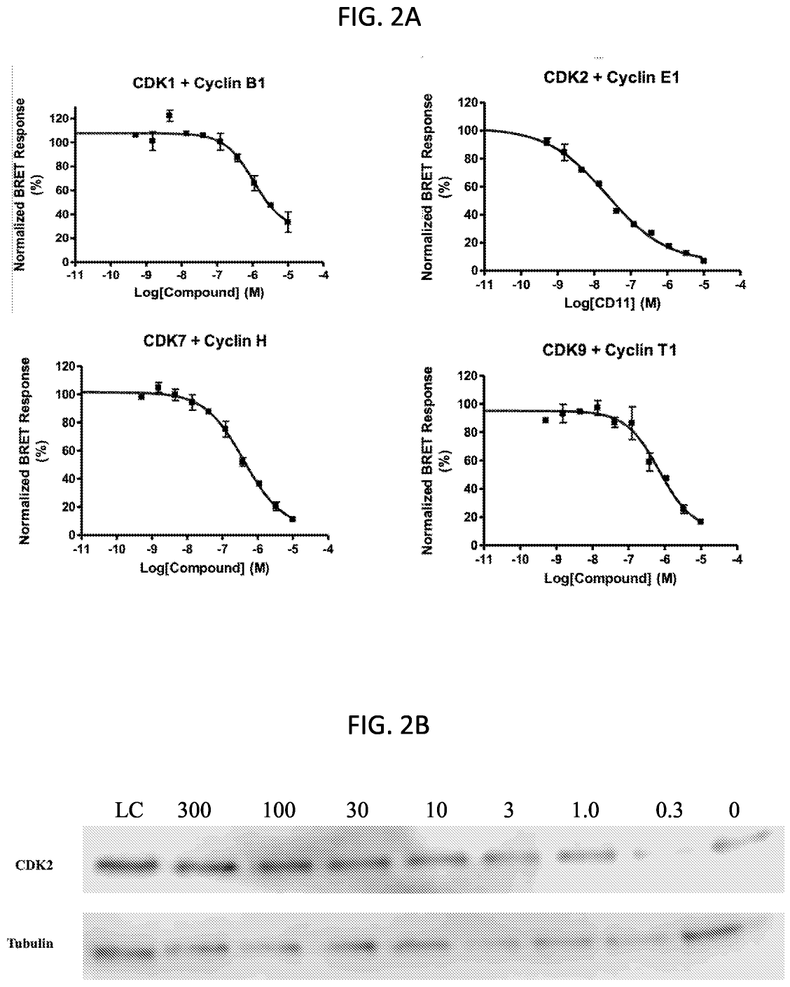

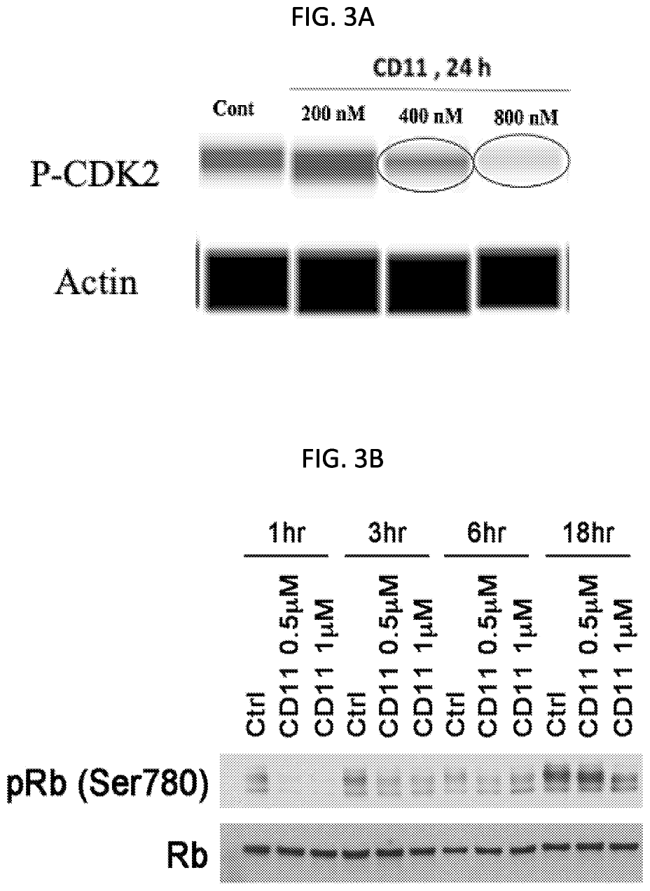

Compound 1-3 efficacy was assessed in several in vitro assays. A nanoBrET assay was used to measure intracellular kinase activity of I-3 with CDK2, CDK1, CDK7 and CDK9. Briefly, HEK293 cells were transfected with CDK2, CDK1, CDK7 and CDK9. The transfected cells were treated in duplicate with test compound I-3 (starting at 10 μM, 10-dose with 3-fold dilution) and target engagement was measured by NanoBRET assay (FIG. 2A and Table 4). Cellular thermal shift assay (CETSA) was also performed to evaluate drug target interaction in the cells. The CETSA method is built upon two concepts: cellular thermal shift and isothermal dose-response. The cellular thermal shift assay is based on the established fact that proteins that are complexed to a ligand become more resistant to heat induced unfolding. Isothermal dose response is used to determine a compound's potency in binding the target by measuring the thermal stability of the target treated with various concentrations of compound at a fixed temperature. Briefly, cell lysate was treated with I-3 (starting at 300 nM, 10-dose with 3-fold dilution) and incubated for 1 hour and cell lysates were then subjected to predetermined thermal aggregation temperature (58° C.) for another 20 min. Western blot was performed to determine the levels of target protein bound to I-3 (FIG. 2B). The resulting data in both drug target engagement methods demonstrates potent binding (nanomolar range) of CDK2/Cyclin E. In addition, increasing amounts of I-3 reduced pCDK2 and phosphor-RB levels in MDA-MB-468 cells (FIGS. 3A and 3B).

TABLE 4
NanoBRET target engagement assay results

| | IC₅₀ (nM) |
|---|---|
| CDK1/Cyclin B1 | 1070 |
| CDK2/Cyclin E1 | 22 |
| CDK9/Cyclin T1 | 702 |
| CDK7/Cyclin H | 403 |

Tables 5 and 6 show assessment of in vitro efficacy of I-3 in a panel of breast cancer cell lines. A panel of breast cancer cell lines were treated with I-3 in 10-dose IC₅₀ mode in triplicate with 3-fold serial dilution starting at 10 μM for 72 hours (Table 5). In addition, basal like (MDA-MB-468) and Normal (MCF-10A) breast cancer cell lines were treated with I-3, PF-06873600 and CYC065 in 10-dose IC₅₀ mode in triplicate with 3-fold serial dilution starting at 10 μM for 72 hours (Table 6).

TABLE 5
Cell viability in breast cancer cell lines treated with I-3

| | Molecular | CTG and Alamar Blue |
|---|---|---|
| Cell Line | Signature | Assay IC₅₀ (μM) |
| BT-549 | Mesenchymal | 0.765 |
| MDA-MB-231 | Mesenchymal-like | 0.565 |
| MDA-MB-468 | Basal-like 1 | 0.276 |
| T47D | Ductal carcinoma | 55.5 |
| MCF-10A | Normal breast-like | 2.07 |

TABLE 6
Cell viability by CellTiter Glo assay in cell lines
treated with I-3 and other CDK inhibitors.

| | Molecular | | IC50 (μM) | | |
|---|---|---|---|---|---|
| Cell Line | Signature | Mutations | I-3 | PF-06873600 | CYC065 |
| MDA- | Basal- | PTEN; | 0.276 | 0.065 | 0.38 |
| MB-468 | like 1 | RB1; | | | |
| | | SMAD4; | | | |
| | | TP53 | | | |
| MCF- | Normal- | | 2.07 | 0.66 | 0.40 |
| 10A | like Breast | | | | |
| | epithelial | | | | |

| | | | |
|---|---|---|---|
| Safety window (Normal/cancer) | 10 | 10 | 1 |

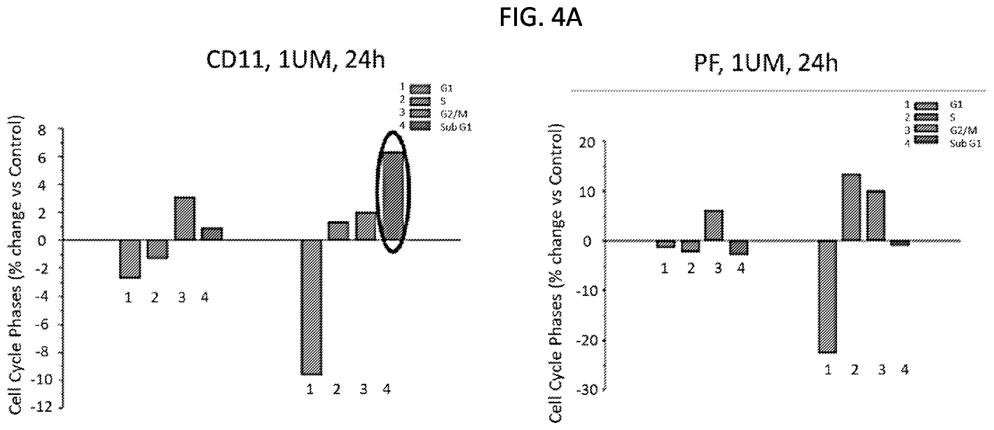

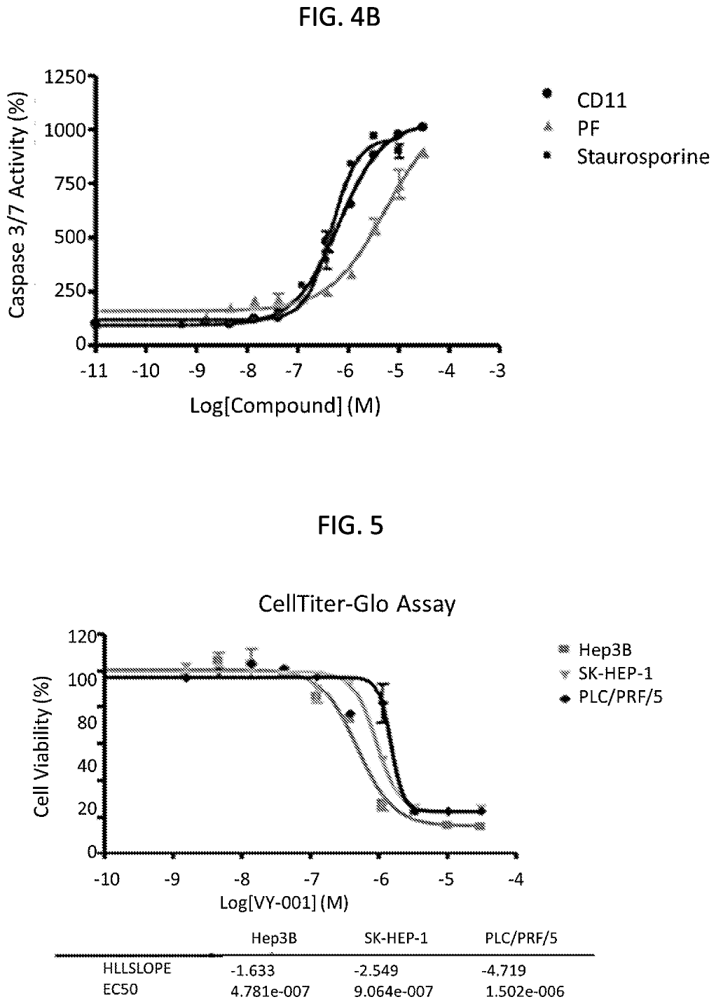

Further anti-proliferative effect of 1-3 was evaluated in the TNBC cell line MDA-MB-468 based on biomarker (CDK2) inhibition. MDA-MB-468 cell lines were treated with I-3 or PF-06873600 for 24 hours at 1 μM, then fixed and stained for cell cycle analysis by propidium iodide (FIG. 4A). The mechanistic cell cycle block is aligned with biomarker. In addition, Caspase Glo 3/7 activation was tested to confirm the sub-G1 (apoptotic) arrest in cell cycle. MDA-MB-468 cell lines were treated with I-3, PF-06873600, or staurosporine control in a 10-dose IC₅₀ mode in triplicate with 3-fold serial dilution starting at 10 μM for 24 hours. The data demonstrates the clear activation of caspase 3/7 by C-11 and no caspase 3/7 activation by PF-06873600 (FIG. 4B and Table 7).

TABLE 7
Caspase Glo 3/7 activity assay.

| Agent | IC₅₀ (μM) |
|---|---|
| I-3 | 0.652 |
| PF-06873600 | 5.66 |

I-3 was also tested for efficacy on hepatocellular carcinoma (HCC) cell lines. Cell viability was measured by CellTiter Glo assay (FIG. 5 and Table 8). Caspase Glo 3/7 was also assessed in PLC/PRF/5 cells, demonstrating greater activation of caspase 3/7 by 1-3 than by PF-06873600 (Table 9).

TABLE 8
Cell viability of PLC/PRF/5 cells

| Test Agent | IC50 (μM) |
|---|---|
| I-3 | 1.52 |
| PF-06873600 | 3.48 |
| Sorafenib | 6.30 |

TABLE 9
Caspase Glo 3/7 activity assay in PLC/PRF/5 cells

| Agent | IC₅₀ (μM) |
|---|---|
| I-3 | 2.45 |
| PF-06873600 | 3.56 |

### Example 9

Evaluation of pharmacokinetic (PK) parameters and efficacy of I-3 in a xenograft model was carried out in mice. The snapshot plasma pharmacokinetics of I-3, merioline-3, and PF-06873600 administered through the intravenous (IV) and per-oral (PO) route were evaluated. Briefly, mice were treated with 5 mg/kg of 1-3 or merioline-3. Plasma was then collected at different time points and measured for drug concentration. The results are summarized in Tables 10 and 11. These data suggest that I-3 has better PK parameters (T_{1/2} and AUC) in both IV and PO administration, compared to merioline-3 and PF-06873600.

TABLE 10
Comparison of plasma PK of I-3 and Merioline-3

| | | |
|---|---|---|
| Parameter | I-3 | Merioline-3 |

IV (5 mg/kg)

| | | |
|---|---|---|
| Tmax (min) | 5.0 | 5.0 |
| Cmax (ng/mL) | 5080 | 5560 |
| T_{1/2} (min) | 69.1 | 7.45 |
| AUC_{inf} (min*ng/mL) | 222,000 | 83,600 |

PO (5 mg/kg)

| | | |
|---|---|---|
| Tmax (min) | 30.0 | 60.0 |
| Cmax (ng/mL) | 178 | 483 |
| T_{1/2} (min) | 186 | ND |
| AUC_{inf} (min*ng/mL) | 89,100 | 4340 |

TABLE 11
Comparison of plasma PK of I-3 and other CDK
inhibitors (PO 5 mg/kg, cassette dosing)

| | Parameter | Plasma |
|---|---|---|
| I-3 | Cmax (ng/mL) | 483 |
| | T_{1/2} (h) | 3.1 |
| | AUC_{inf} (min*ng/mL) | 1866 |
| Merioline-3 | Cmax (ng/mL) | 178 |
| | T_{1/2} (h) | ND |
| | AUC_{inf} (min*ng/mL) | ND |
| PF-06873600 | Cmax (ng/mL) | 196 |
| | T_{1/2} (h) | 2.1 |
| | AUC_{inf} (min*ng/mL) | 641 |

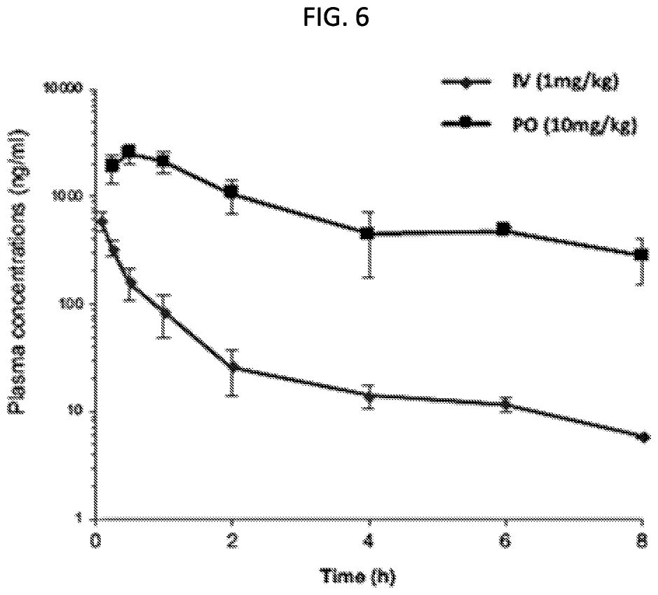

Absolute bioavailability of I-3 was assessed after IV and PO dosing in CD-1 male mice. Mean plasma concentration (FIG. 6) and PK parameters (Table 12) are shown. After IV administration, I-3 exhibited mono-phasic decline. Plasma concentrations were observed up to 8 hours, and plasma concentrations were not observed at 24 hour time point. Volume of distribution (12.5 L/kg) was 17-fold higher compared to total body water content (0.7 L/kg), with high half-life. Plasma clearance (2.5 L/kg) was moderate. This is approximately 50% of the hepatic blood flow (5.4 L/kg) in mice. I-3 exhibited rapid oral absorption (Tₘₐₓ of 0.5-1 hour) with mean plasma Cₘₐₓ of 23-4 ng/mL and systemic exposures of AUC0-t of 5667 h*ng/mL. Oral suspension bioavailability (% F) was >100%, indicating saturation of elimination pathways at the dose of 10 mg/kg.

TABLE 12
PK parameters of I-3 in male CD1 mice

| | IV | PO |
|---|---|---|
| Parameter | (1 mg/kg) | (10 mg/kg) |
| Cₘₐₓ (ng/mL) | NA | 2504.8 ± 55.5 |
| Tₘₐₓ (h) | NA | 1.0 ± 0.3 |
| AUC₍₀₋ₜ₎ (hr*ng/mL) | 384.2 ± 98.9 | 5667.5 ± 568.7 |
| AUC_{(inf)} (hr*ng/mL) | 2.2 ± 0.7 | 6570.1 ± 541.7 |
| MRT_{(inf)} (h) | | NA |
| C_{Lp} (L/hr/kg) | 2.5 ± 0.6 | NA |
| Vdₛₛ (L/kg) | 6.8 ± 2.4 | NA |
| T_{1/2} (h) | 3.4 ± 0.7 | NA |
| Bioavailability (% F) | NA | 147.5 ± 14.8 |

PK parameters of I-3 was also compared to a panel of CDK inhibitors (Table 13). While Milciclib has a better Cₘₐₓ and AUC_{inf} than I-3, it is a poly-CDK inhibitor and is not CDK2-specific. Likewise, Ribociclib has a better Cₘₐₓ than I-3, but it is primarily a CDK4/6 inhibitor.

TABLE 13
Comparison of in vivo PK of CDK inhibitors

| Parameter | I-3 | Palbociclib | Milciclib | Abemaciclib | Ribociclib |
|---|---|---|---|---|---|
| Cₘₐₓ | 483 | 196 | 537 | 107 | 303 |
| (ng/mL) | | | | | |
| Tₘₐₓ | 3.1 | 2.1 | 3.5 | 5.5 | 5.7 |
| (hr) | | | | | |
| AUC_{inf} | 1866 | 641 | 2983 | 613 | 2783 |
| (hr*ng/mL) | | | | | |

Anti-tumor efficacy of I-3 was evaluated in a MDA-MB-231 breast cancer xenograft model. Briefly, mice with MDA-MB-231 tumors were randomized to three treatment groups: control (Vehicle, n=8), 1-3 IV (10 mg/kg, n=8) and 1-3 PO (10 mg/kg, n=8) on a schedule of 14 days for daily (PO) or twice weekly (IV). Oral gavage treatments were formulated in 0.1% Tween 80 and 99.9% of 0.5% (w/v) of methyl cellulose in water. Intravenous treatments were formulated in 5% (v/v) DMSO, 2.5% (v/v) absolute ethanol, 2.5% (v/v) Solutol, and 90% Normal saline. Tumor growth was measured twice weekly by using a digital vernier caliper and high resolution images of tumor bearing mice were captured using a digital camera at weekly intervals of the study (Days 0, 7 and 14). Tumor volume was calculated as follows:

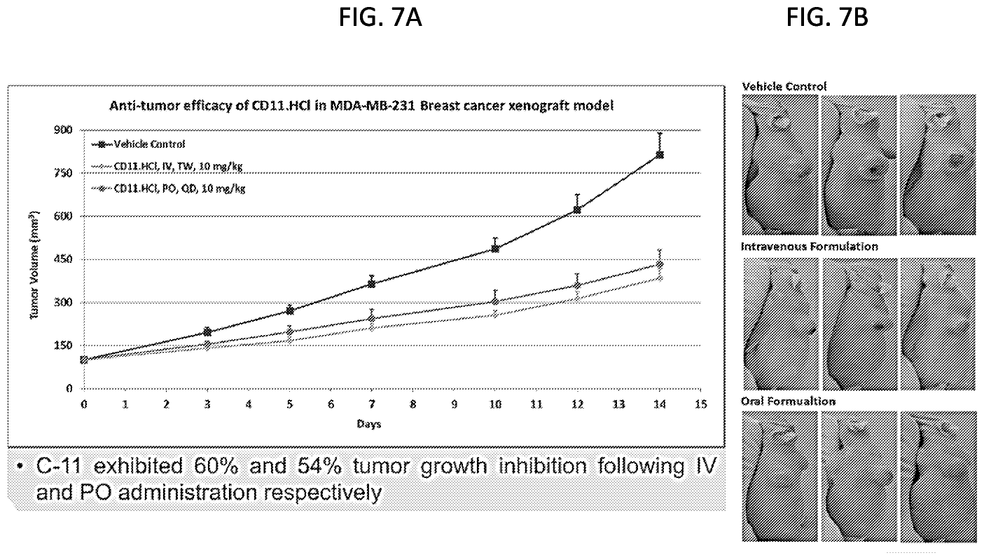

Tumor Volume=[Length(L)×Width(W)²]/2

where length (L) is the largest diameter of the tumor and width (W) is the smallest diameter of the tumor. The efficacy of the test compound was assessed in terms of tumor growth inhibition (TGI) compared to control group (FIGS. 7A and 7B).

Twice weekly IV administration of I-3 at 10 mg/kg resulted in a significant (p<0.0001) tumor growth inhibition (TGI) of 60% compared to the control group. Once daily oral administration of 1-3 at 10 mg/kg resulted in a significant (p<0.0001) 54% TGI at the end of two weeks. At the end of the study, blood samples were collected from the animals for analysis of complete blood counts (CBC) using an automated hematology analyser (ADVIA2120, Siemens Ltd.). There were no significant differences in complete blood counts between the treatment and control group. Once daily oral doses of 10 mg/Kg and twice weekly IV doses of 10 mg/kg were well-tolerated by the animals during the course of the treatment, with no changes in body weight or blood counts compared to vehicle control over the course of the study (Tables 14 and 15).

TABLE 14
In vivo safety of I-3 - body weight

| | |
|---|---|
| Dose | Body Weight (g), mean ± SE |

| Test Group | (mg/kg) | Day 0 | Day 7 | Day 14 |
|---|---|---|---|---|
| Vehicle | 0 | 22.0 ± 0.3 | 21.6 ± 0.7 | 22.6 ± 0.9 |
| Control | | | | |
| IV | 10 | 22.1 ± 0.6 | 22.3 ± 1.0 | 22.9 ± 0.7 |
| PO | 10 | 21.6 ± 0.4 | 20.9 ± 0.9 | 21.6 ± 0.9 |

TABLE 15
In vivo safety of I-3 - blood count

| | Treatment Group (Mean ± SE) | | |
|---|---|---|---|
| | Vehicle | IV | PO |
| Parameter | Control | (10 mg/kg) | (10 mg/kg) |
| White blood cells | 32.0 ± 4.1 | 22.9 ± 8.3 | 36.6 ± 6.8 |
| (10³ cells/μL) | | | |
| Red blood cells | 7.7 ± 0.2 | 8.4 ± 0.5 | 6.9 ± 0.2 |
| (10⁶ cells/μL) | | | |
| Hemoglovin (g/dL) | 11.8 ± 0.3 | 12.8 ± 0.8 | 10.6 ± 0.3 |
| Hematocrit (%) | 38.8 ± 0.8 | 38.4 ± 3.7 | 34.2 ± 0.9 |
| Mean corpuscular | 46.6 ± 3.8 | 50.2 ± 0.3 | 44.8 ± 4.1 |
| volume (fL) | | | |
| Mean corpuscular | 15.3 ± 0.1 | 15.2 ± 0.2 | 15.3 ± 0.1 |
| hemoglobin (pg) | | | |
| Mean corpuscular | 30.5 ± 0.1 | 30.3 ± 0.2 | 31.1 ± 0.2 |
| hemoglobin | | | |
| concentration (g/dL) | | | |
| Platelets | 899.5 ± 146.0 | 802.4 ± 107.9 | 762.1 ± 99.3 |
| (10³ cells/μL) | | | |

In view of the many possible embodiments to which the principles of the disclosure may be applied, it should be recognized that the illustrated embodiments are only examples of the disclosed technology and should not be taken as limiting the scope of the technology. Rather, the scope of the disclosed technology is defined by the following claims. I therefore claim as the disclosed technology all that comes within the scope and spirit of these claims.

1. A compound according to Formula I 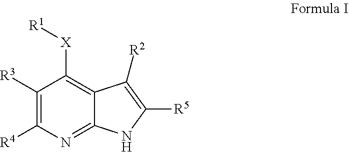
or a pharmaceutically acceptable salt thereof, wherein:
R¹ is phenyl optionally substituted with one or more substituents;
X is O;
R² is a 5- or 6-membered nitrogen-containing heteroaryl substituted with NR′₂ or OH and optionally further substituted with one or more additional substituents;
each R′ independently is H, C₁₋₆alkyl or —C(O)CH₂N(C₁₋₆alkyl)₂, and
each of R³, R⁴ and R⁵ independently is H or aliphatic.

2. The compound according to claim 1, wherein the compound has a formula 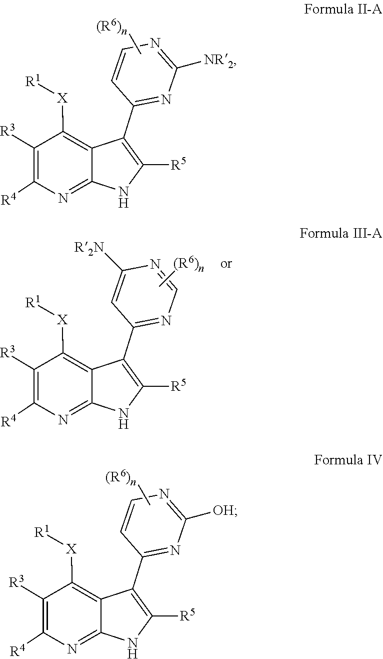
or a pharmaceutically acceptable salt thereof, wherein:
n is 0, 1, or 2;
each R⁶ independently is amino, OH, halogen, alkyl, haloalkyl, CO₂H, CO₂R, NO₂, CN, amide, sulfonic acid, sulfonamide, hydroxyalkyl, alkoxy, or heteroaliphatic, where R is aliphatic; and
each R′ independently is H, C₁₋₆alkyl or —C(O)CH₂N(C₁₋₆alkyl)₂.

3. The compound according to claim 2, wherein the compound has a formula

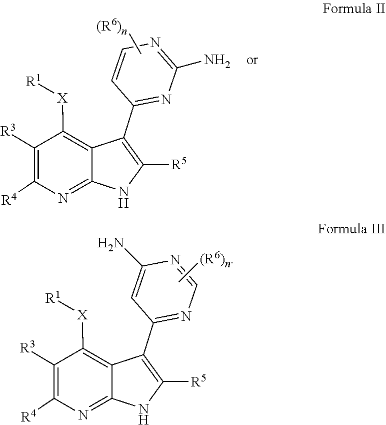

4. The compound according to claim 1, wherein the compound has a formula 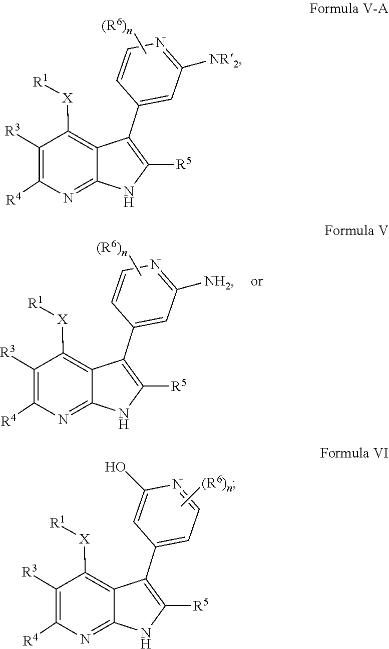
or a pharmaceutically acceptable salt thereof, wherein:
n is 0, 1, or 2;
each R⁶ independently is amino, OH, halogen, alkyl, haloalkyl, CO₂H, CO₂R, NO₂, CN, amide, sulfonic acid, sulfonamide, hydroxyalkyl, alkoxy, or heteroaliphatic, where R is aliphatic; and
each R′ independently is H, C₁₋₆alkyl or —C(O)CH₂N(C₁₋₆alkyl).

5-7. (canceled)

8. The compound according to claim 2, wherein the compound has a formula 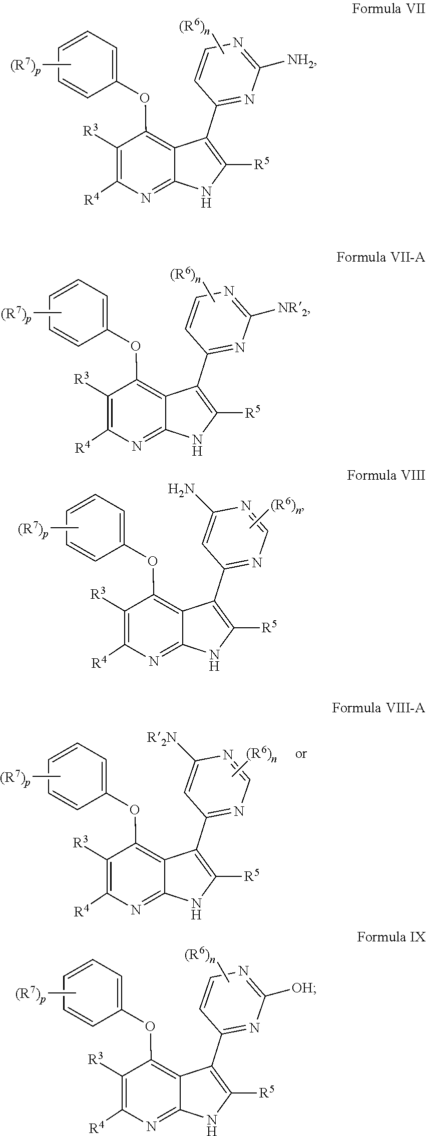
or a pharmaceutically acceptable salt thereof, wherein:
p is from 0 to 5; and
each R⁷ independently is amino, OH, halogen, alkyl, haloalkyl, CO₂H, CO₂R, NO₂, CN, amide, sulfonic acid, sulfonamide, hydroxyalkyl, alkoxy, or heteroaliphatic, where R is aliphatic.

9. The compound according to claim 8, wherein R³, R⁴ and R⁵ are H.

10. The compound according to claim 8, wherein n is 0.

11. The compound according to claim 1, wherein the compound is a salt.

12. The compound according to claim 11, wherein the compound is a hydrochloride salt.

13. The compound of claim 1, selected from:
I-1: 4-(4-phenoxy-1H-pyrrolo[2,3-b]pyridin-3-yl)pyrimidin-2-amine;
I-2: 4-(4-(3-chlorophenoxy)-1H-pyrrolo[2,3-b]pyridin-3-yl)pyrimidin-2-amine;
I-3: 4-(4-(3-fluorophenoxy)-1H-pyrrolo[2,3-b]pyridin-3-yl)pyrimidin-2-amine;
I-4: 4-(4-(3,5-difluorophenoxy)-1H-pyrrolo[2,3-b]pyridin-3-yl)pyrimidin-2-amine;
I-5: 4-(4-(3-(trifluoromethyl)phenoxy)-1H-pyrrolo[2,3-b]pyridin-3-yl)pyrimidin-2-amine;
I-6: 4-(4-(4-fluorophenoxy)-1H-pyrrolo[2,3-b]pyridin-3-yl)pyrimidin-2-amine;
I-7: 4-(4-phenoxy-1H-pyrrolo[2,3-b]pyridin-3-yl)pyrimidin-2-ol;
I-8: 4-(4-(3-chlorophenoxy)-1H-pyrrolo[2,3-b]pyridin-3-yl)pyrimidin-2-ol;
I-9: 4-(4-(3-fluorophenoxy)-1H-pyrrolo[2,3-b]pyridin-3-yl)pyrimidin-2-ol;
I-10: 4-(4-(3,5-difluorophenoxy)-1H-pyrrolo[2,3-b]pyridin-3-yl)pyrimidin-2-ol;
I-11: 4-(4-(3-(trifluoromethyl)phenoxy)-1H-pyrrolo[2,3-b]pyridin-3-yl)pyrimidin-2-ol;
I-12: 4-(4-(4-fluorophenoxy)-1H-pyrrolo[2,3-b]pyridin-3-yl)pyrimidin-2-ol;
I-13: 6-(4-phenoxy-1H-pyrrolo[2,3-b]pyridin-3-yl)pyrimidin-4-amine;
I-14: 6-(4-(3-chlorophenoxy)-1H-pyrrolo[2,3-b]pyridin-3-yl)pyrimidin-4-amine;
I-15: 6-(4-(3-fluorophenoxy)-1H-pyrrolo[2,3-b]pyridin-3-yl)pyrimidin-4-amine;
I-16: 6-(4-(3,5-difluorophenoxy)-1H-pyrrolo[2,3-b]pyridin-3-yl)pyrimidin-4-amine;
I-17: 6-(4-(3-(trifluoromethyl)phenoxy)-1H-pyrrolo[2,3-b]pyridin-3-yl)pyrimidin-4-amine;
I-18: 6-(4-(4-fluorophenoxy)-1H-pyrrolo[2,3-b]pyridin-3-yl)pyrimidin-4-amine;
I-19: 4-(4-phenoxy-1H-pyrrolo[2,3-b]pyridin-3-yl)pyrimidin-2-amine hydrochloride;
I-20: 4-(4-(3-chlorophenoxy)-1H-pyrrolo[2,3-b]pyridin-3-yl)pyrimidin-2-amine hydrochloride;
I-21: 4-(4-(3-fluorophenoxy)-1H-pyrrolo[2,3-b]pyridin-3-yl)pyrimidin-2-amine hydrochloride;
I-22: 4-(4-(3,5-difluorophenoxy)-1H-pyrrolo[2,3-b]pyridin-3-yl)pyrimidin-2-amine hydrochloride;
I-23: 4-(4-(3-(trifluoromethyl)phenoxy)-1H-pyrrolo[2,3-b]pyridin-3-yl)pyrimidin-2-amine hydrochloride;
I-24: 4-(4-(4-fluorophenoxy)-1H-pyrrolo[2,3-b]pyridin-3-yl)pyrimidin-2-amine hydrochloride;
I-25: 4-(4-phenoxy-1H-pyrrolo[2,3-b]pyridin-3-yl)pyrimidin-2-ol hydrochloride;
I-26: 4-(4-(3-chlorophenoxy)-1H-pyrrolo[2,3-b]pyridin-3-yl)pyrimidin-2-ol hydrochloride;
I-27: 4-(4-(3-fluorophenoxy)-1H-pyrrolo[2,3-b]pyridin-3-yl)pyrimidin-2-ol hydrochloride;
I-28: 4-(4-(3,5-difluorophenoxy)-1H-pyrrolo[2,3-b]pyridin-3-yl)pyrimidin-2-ol hydrochloride;
I-29: 4-(4-(3-(trifluoromethyl)phenoxy)-1H-pyrrolo[2,3-b]pyridin-3-yl)pyrimidin-2-ol hydrochloride;
I-30: 4-(4-(4-fluorophenoxy)-1H-pyrrolo[2,3-b]pyridin-3-yl)pyrimidin-2-ol hydrochloride;
I-31: 6-(4-phenoxy-1H-pyrrolo[2,3-b]pyridin-3-yl)pyrimidin-4-amine hydrochloride;
I-32: 6-(4-(3-chlorophenoxy)-1H-pyrrolo[2,3-b]pyridin-3-yl)pyrimidin-4-amine hydrochloride;
I-33: 6-(4-(3-fluorophenoxy)-1H-pyrrolo[2,3-b]pyridin-3-yl)pyrimidin-4-amine hydrochloride;
I-34: 6-(4-(3,5-difluorophenoxy)-1H-pyrrolo[2,3-b]pyridin-3-yl)pyrimidin-4-amine hydrochloride;
I-35: 6-(4-(3-(trifluoromethyl)phenoxy)-1H-pyrrolo[2,3-b]pyridin-3-yl)pyrimidin-4-amine hydrochloride;
I-36: 6-(4-(4-fluorophenoxy)-1H-pyrrolo[2,3-b]pyridin-3-yl)pyrimidin-4-amine hydrochloride;
I-37: 4-(4-(3-fluorophenoxy)-1H-pyrrolo[2,3-b]pyridin-3-yl)pyrimidin-2-amine;
I-38: 4-(4-(3-fluoro-5-(morpholinomethyl)phenoxy)-1H-pyrrolo[2,3-b]pyridin-3-yl)pyrimidin-2-amine;
I-39: 4-(4-(3-((dimethylamino)methyl)-5-fluorophenoxy)-1H-pyrrolo[2,3-b]pyridin-3-yl)pyrimidin-2-amine; or
I-40: 2-(dimethylamino)-N-(4-(4-(3-fluorophenoxy)-1H-pyrrolo[2,3-b]pyridin-3-yl)pyrimidin-2-yl)acetamide.

14. A pharmaceutical composition, comprising a compound according to claim 1 and a pharmaceutically acceptable excipient.

15. A method of treating a subject with cancer, comprising administering a compound according to claim 1 to a subject in need thereof.

16. The method of claim 15, wherein the subject is human.

17. The method of claim 16, wherein the cancer is breast cancer, ovarian cancer, pancreatic cancer, or hepatocellular carcinoma.

18. The method of claim 17, wherein the breast cancer is triple negative breast cancer.

19. The method of claim 15, wherein the cancer expresses or overexpresses CDK2.

20. A method for reducing or inhibiting activity or expression of CDK2, comprising contacting a cell with an effective amount of a compound according to claim 1.

21. The method of claim 20, wherein the cell is in a human or non-human animal subject.

22. A compound selected from:
I-41: 4-(4-((5-fluoropyridin-3-yl)oxy)-1H-pyrrolo[2,3-b]pyridin-3-yl)pyrimidin-2-amine;
I-42: 4-(4-(piperidin-4-yloxy)-1H-pyrrolo[2,3-b]pyridin-3-yl)pyrimidin-2-amine; or
I-43: 4-(4-((4-fluorobicyclo[1.1.1]pentan-2-yl)oxy)-1H-pyrrolo[2,3-b]pyridin-3-yl)pyrimidin-2-amine;
or a pharmaceutically acceptable salt thereof.

23. A pharmaceutical composition, comprising a compound according to claim 22 and a pharmaceutically acceptable excipient.

24. A method of treating a subject with cancer, comprising administering a compound according to claim 22, to a subject in need thereof.
